(19) United States
(12) Patent Application Publication
(10) Pub. No.: US 2015/0174144 A1
(43) Pub. Date: Jun. 25, 2015

(54) TETRACYCLINE COMPOUNDS FOR TREATING NEURODEGENERATIVE DISORDERS

(71) Applicant: PARATEK PHARMACEUTICALS, INC., Boston, MA (US)

(72) Inventors: Todd Bowser, Charlton, MA (US); Paul Higgins, Danvers, MA (US); Michael P. Draper, Windham, NH (US); S. Ken Tanaka, Bellevue, WA (US)

(21) Appl. No.: 14/414,567

(22) PCT Filed: Jul. 15, 2013
(86) PCT No.: PCT/US2013/050495

§ 371 Date: Jan. 13, 2015

Related U.S. Application Data
(60) Provisional application No. 61/671,587, filed on Jul. 13, 2012.

(51) Int. Cl. A61K 31/65

(52) U.S. Cl. CPC A61K 31/65

(57) ABSTRACT

Tetracycline compounds for treating neurodegenerative disorders are disclosed herein. Also disclosed is a pharmaceutical composition comprising the tetracycline compounds, and a method for treating, preventing, or ameliorating neurodegenerative disorders or inflammation in a subject by administering the tetracycline compounds or a pharmaceutical composition thereof, either alone or in combination with a second therapeutic agent.

### RELATED APPLICATIONS

This application claims priority to U.S. Provisional Application No. 61/671,587, filed on Jul. 13, 2012, the entire contents of which are hereby incorporated herein by reference

### BACKGROUND OF THE INVENTION

A large body of scientific literature has shown the neuroprotective characteristics of tetracyclines. Tetracyclines, such as minocycline, have been shown to suppress microglial activation, inhibit apoptosis of neuronal cells after glutamate excitotoxicity, scavenge reactive oxygen and nitrogen species, and suppress matrix metalloproteinase (MMP) activity. Tetracyclines have also shown efficacy as a neuroprotective agent in animal models of stroke, Huntingdon's disease, Parkinson's disease, ALS, Alzheimer's disease, and spinal cord injury. Clinically, tetracyclines have been effective at improving clinical outcome after acute ischemic stroke, and are currently being evaluated in trials of Parkinson's disease, spinal cord injury, schizophrenia, and other neurodegenerative diseases.

Clinical studies of tetracyclines have demonstrated very favorable therapeutic efficacy through the significant reduction of central nervous system (CNS) lesions and an improvement of EDSS scores comparable to or better than clinically-approved multiple sclerosis (MS) treatments. Additional studies have shown tetracyclines to further benefit MS patients, such as minocycline, when used in combination with COPAXONE® and, doxycycline, when used in combination with AVONEX®. Tetracyclines have also been effective in treating MS in animal models. In experimental autoimmune encephalomyelitis (EAE), minocycline exhibited a positive effect on disease course, either alone or in combination with other drugs such as glatiramer acetate and IFN. Tetracyclines were also effective at increasing survival of retinal ganglion cells in a rat model of MOG-induced optic neuritis.

Unlike the current immune-modulating treatments, tetracyclines also have demonstrated efficacy as neuroprotectants, and have the unique potential to effectively limit progressive neurodegeneration, e.g., such as seen in all forms of MS. Although promising as MS treatments in their own right, the clinically-used tetracyclines are broad-spectrum antibiotics which may cause gastrointestinal upset, opportunistic fungal infections, and the development of bacterial resistance after chronic use. In addition, several of the tetracyclines are known to cause undesirable photosensitivity reactions and tissue staining. Thus, there is currently a need to develop novel tetracycline compounds designed specifically for the chronic treatment of MS and other neurodegenerative disorders by removing the antibacterial activity of the compound while maintaining or improving the long-established safety, pharmacokinetics, and efficacy of the tetracycline class. The present invention addresses this need.

### SUMMARY OF THE INVENTION

The present invention relates to a compound of formula (I), (Ia) or (Ib):

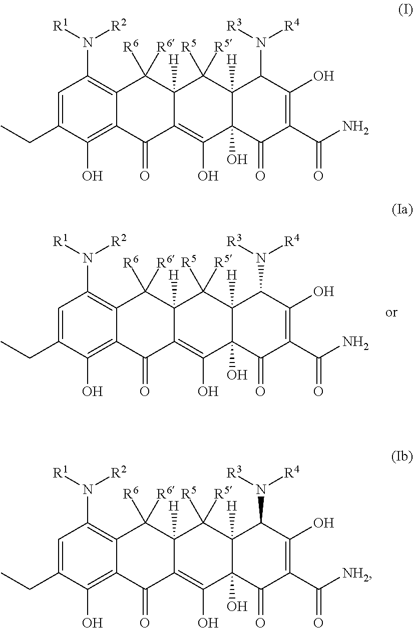

or a pharmaceutically acceptable salt thereof, wherein:

R¹, R², R³ and R⁴ are each independently H or unsubstituted C₁-C₆ alkyl; and

R⁵, R^{5′}, R⁶ and R^{6′} are each independently H, hydroxyl, or unsubstituted C₁-C₆ alkyl.

The present invention also relates to a pharmaceutical composition comprising a tetracycline compound of formula (I), (Ia) or (Ib) and a pharmaceutically acceptable carrier. Such a pharmaceutical composition can be used in treating, preventing, or ameliorating a neurodegenerative disease.

The present invention also relates to a pharmaceutical composition comprising a tetracycline compound of formula (I), (Ia) or (Ib) and a pharmaceutically acceptable carrier. Such a pharmaceutical composition can be used in treating, preventing, or ameliorating multiple sclerosis.

The present invention also relates to a method for treating, preventing, or ameliorating a neurodegenerative disease in a subject. The method includes administering to the subject an effective amount of a tetracycline compound of formula (I), (Ia) or (Ib) or a pharmaceutical composition thereof, such that the neurodegenerative disease is treated, prevented, or ameliorated.

The present invention also relates to a method for treating, preventing, or ameliorating multiple sclerosis in a subject. The method includes administering to the subject an effective amount of a tetracycline compound of formula (I), (Ia) or (Ib) or a pharmaceutical composition thereof, such that multiple sclerosis is treated, prevented, or ameliorated.

### BRIEF DESCRIPTION OF THE FIGURES

FIG. 1 shows the effect of minocycline and Compound 1 on the clinical course of MOG peptide-induced EAE in B57BL/6 mice.

FIG. 2 shows the effect of minocycline and Compound 1 on the clinical course of rat EAE.

FIG. 3 shows the effect of Compound 1 on the clinical course of mouse EAE after oral administration.

FIG. 4 shows the effect of Compound 1 on the clinical course of rat EAE after oral administration.

FIG. 5 is a dose response of the inhibition of glutamate-induced neurodegeneration in cerebellar granule neurons by minocycline and Compound 1.

FIG. 6 shows sample brain slices from 90 min temporary MCA occluded rats stained with TTC.

FIG. 7 shows the in vitro effect of minocycline and Compound 1 in the cell-free MMP-9 activity assay.

FIG. 8 shows the in vitro effect of minocycline and Compound 1 on the LPS-induced production of NO by J774A.1 murine macrophages.

FIG. 9 shows the in vitro effect of minocycline and Compound 1 on the LPS-induced production of TNFα by RAW 264.7 murine macrophages.

FIG. 10 shows the time course of EA-Trolox oxidation, with broken lines indicating 50% degradation mark.

FIG. 11 shows the time spent in the center during the Elevated Plus Maze test in the mouse model of Fragile X Syndrome and in wild-type mice after treatment with Compound 1 and the negative control.

FIG. 12 shows the time spent in the close arm during the Elevated Plus Maze test in the mouse model of Fragile X Syndrome and in wild-type mice after treatment with Compound 1 and the negative control.

FIG. 13 shows the results of trial 1 of the Open Field test in the mouse model of Fragile X Syndrome and in wild-type mice after treatment with Compound 1 and the negative control.

FIG. 14 shows the results of trial 2 of the Open Field test in the mouse model of Fragile X Syndrome and in wild-type mice after treatment with Compound 1 and the negative control.

FIG. 15 shows the results of trial 3 of the Open Field test in the mouse model of Fragile X Syndrome and in wild-type mice after treatment with Compound 1 and the negative control.

### DETAILED DESCRIPTION OF THE INVENTION

The present invention relates to a compound of formula (I), (Ia) or (Ib):

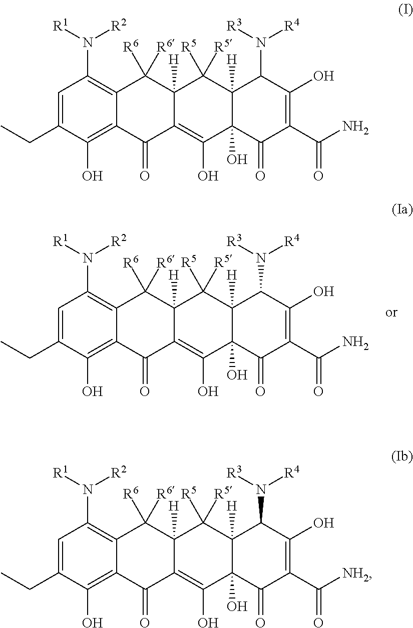

or a pharmaceutically acceptable salt thereof, wherein:

R¹, R², R³ and R⁴ are each independently H or unsubstituted C₁-C₆ alkyl; and

R⁵, R^{5′}, R⁶ and R^{6′} are each independently H, hydroxyl, or unsubstituted C₁-C₆ alkyl.

In one embodiment, R¹, R², R³ and R⁴ are each methyl, ethyl, n-propyl, i-propyl, n-butyl, t-butyl, pentyl, or hexyl. In a preferred embodiment, R¹, R², R³ and R⁴ are each methyl.

In one embodiment, R⁵, R^{5′}, R⁶ and R^{6′} are each hydrogen. In another embodiment, R⁵ and R^{5′} are each hydrogen; and one of R⁶ and R^{6′} is hydroxyl and the other is methyl. In another embodiment, one of R⁵ and R^{5′} is hydrogen and the other is hydroxyl; and one of R⁶ and R^{6′} is hydrogen and the other is methyl. In another embodiment, one of R⁵ and R^{5′} is hydrogen and the other is hydroxyl; and one of R⁶ and R^{6′} is hydroxyl and the other is methyl.

In one embodiment, R¹, R², R³ and R⁴ are each methyl; R⁵, R^{5′}, R⁶ and R^{6′} are each hydrogen. In another embodiment, R¹, R², R³ and R⁴ are each methyl; R⁵ and R^{5′} are each hydrogen; and one of R⁶ and R^{6′} is hydroxyl and the other is methyl. In another embodiment, R¹, R², R³ and R⁴ are each methyl; one of R⁵ and R^{5′} is hydrogen and the other is hydroxyl; and one of R⁶ and R^{6′} is hydrogen and the other is methyl. In another embodiment, R¹, R², R³ and R⁴ are each methyl; one of R⁵ and R^{5′} is hydrogen and the other is hydroxyl; and one of R⁶ and R^{6′} is hydroxyl and the other is methyl.

In one embodiment, the tetracycline compound of the present invention is Compound 1, having the following structure:

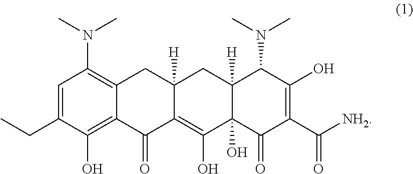 [SMILES: CCc1cc(N(C)C)c2c(c1O)C(=O)C1=C(O)[C@]3(O)C(=O)C(C(N)=O)=C(O)[C@@H](N(C)C)[C@@H]3C[C@@H]1C2]

In another embodiment, the tetracycline compound of the present invention is Compound 2, having the following structure:

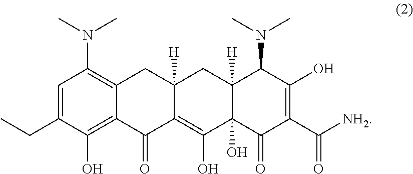 [SMILES: CCc1cc(N(C)C)c2c(c1O)C(=O)C1=C(O)[C@]3(O)C(=O)C(C(N)=O)=C(O)[C@H](N(C)C)[C@@H]3C[C@@H]1C2]

In yet another embodiment, the tetracycline compound of the present invention is Compound 3, having the following structure:

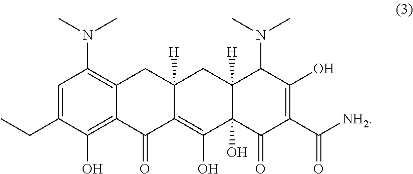 [SMILES: CCc1cc(N(C)C)c2c(c1O)C(=O)C1=C(O)[C@]3(O)C(=O)C(C(N)=O)=C(O)C(N(C)C)[C@@H]3C[C@@H]1C2]

In one embodiment, the tetracycline compounds of the present invention inhibit inflammation at a dosage lower than the dosage of minocycline. In one embodiment, the tetracycline compounds of the present invention inhibit inflammation at a dosage that is approximately 90%, approximately 80%, approximately 70%, approximately 60%, approximately 50%, approximately 40%, approximately 30%, approximately 20%, or approximately 10% of the dosage of minocycline.

In one embodiment, the tetracycline compounds of the present invention, when used at the same dosage as minocycline, show better inhibition of inflammation than minocycline. In one embodiment, the tetracycline compounds of the present invention, when used at the same dosage as minocycline, inhibit approximately 5% more, approximately 10% more, approximately 20% more, approximately 30% more, approximately 40% more, approximately 50% more, approximately 60% more, approximately 70% more, approximately 80% more, approximately 90% more, or approximately 100% more inhibition of inflammation.

In one embodiment, the tetracycline compounds of the present invention inhibit demyelination. In one embodiment, the tetracycline compounds of the present invention inhibit demyelination at a dosage at approximately or less than 100 mg/kg, at approximately or less than 75 mg/kg, at approximately or less than 50 mg/kg, at approximately or less than 40 mg/kg, at approximately or less than 30 mg/kg, at approximately or less than 25 mg/kg, at approximately or less than 20 mg/kg, at approximately or less than 15 mg/kg, at approximately or less than 10 mg/kg, or at approximately or less than 5 mg/kg. In a particular embodiment, the tetracycline compounds of the present invention inhibit demyelination at a dosage at approximately 25 mg/kg.

In one embodiment, the tetracycline compounds of the present invention inhibit demyelination at a dosage lower than the dosage of minocycline. In one embodiment, the tetracycline compounds of the present invention inhibit demyelination at a dosage that is approximately 90%, approximately 80%, approximately 70%, approximately 60%, approximately 50%, approximately 40%, approximately 30%, approximately 20%, or approximately 10% of the dosage of minocycline.

In one embodiment, the tetracycline compounds of the present invention, when used at the same dosage as minocycline, show better inhibition of demyelination than minocycline. In one embodiment, the tetracycline compounds of the present invention, when used at the same dosage as minocycline, inhibit approximately 5% more, approximately 10% more, approximately 20% more, approximately 30% more, approximately 40% more, approximately 50% more, approximately 60% more, approximately 70% more, approximately 80% more, approximately 90% more, or approximately 100% more inhibition of demyelination.

In one embodiment, the tetracycline compounds of the present invention inhibit axon loss. In one embodiment, the tetracycline compounds of the present invention inhibit axon loss at a dosage at approximately or less than 100 mg/kg, at approximately or less than 75 mg/kg, at approximately or less than 50 mg/kg, at approximately or less than 40 mg/kg, at approximately or less than 30 mg/kg, at approximately or less than 25 mg/kg, at approximately or less than 20 mg/kg, at approximately or less than 15 mg/kg, at approximately or less than 10 mg/kg, or at approximately or less than 5 mg/kg. In a particular embodiment, the tetracycline compounds of the present invention inhibit axon loss at a dosage at approximately 25 mg/kg.

In one embodiment, the tetracycline compounds of the present invention inhibit axon loss at a dosage lower the dosage of minocycline. In one embodiment, the tetracycline compounds of the present invention inhibit axon loss at a dosage that is approximately 90%, approximately 80%, approximately 70%, approximately 60%, approximately 50%, approximately 40%, approximately 30%, approximately 20%, or approximately 10% of the dosage of minocycline.

In one embodiment, the tetracycline compounds of the present invention, when used at the same dosage as minocycline, show better inhibition of axon loss than minocycline. In one embodiment, the tetracycline compounds of the present invention, when used at the same dosage as minocycline, inhibit approximately 5% more, approximately 10% more, approximately 20% more, approximately 30% more, approximately 40% more, approximately 50% more, approximately 60% more, approximately 70% more, approximately 80% more, approximately 90% more, or approximately 100% more inhibition of axon loss.

In one embodiment, the tetracycline compounds of the present invention inhibit autoimmune encephalomyelitis. In one embodiment, the tetracycline compounds of the present invention inhibit autoimmune encephalomyelitis at a dosage at approximately or less than 100 mg/kg, at approximately or less than 75 mg/kg, at approximately or less than 50 mg/kg, at approximately or less than 40 mg/kg, at approximately or less than 30 mg/kg, at approximately or less than 25 mg/kg, at approximately or less than 20 mg/kg, at approximately or less than 15 mg/kg, at approximately or less than 10 mg/kg, or at approximately or less than 5 mg/kg. In a particular embodiment, the tetracycline compounds of the present invention inhibit autoimmune encephalomyelitis at a dosage at approximately 60 mg/kg. In a particular embodiment, the tetracycline compounds of the present invention inhibit autoimmune encephalomyelitis at a dosage at approximately 30 mg/kg. In a particular embodiment, the tetracycline compounds of the present invention inhibit autoimmune encephalomyelitis at a dosage at approximately 25 mg/kg. In a particular embodiment, the tetracycline compounds of the present invention inhibit autoimmune encephalomyelitis at a dosage at approximately 15 mg/kg. In a particular embodiment, the tetracycline compounds of the present invention inhibit autoimmune encephalomyelitis at a dosage at approximately 12 mg/kg.

In one embodiment, the tetracycline compounds of the present invention inhibit autoimmune encephalomyelitis at a dosage lower the dosage of minocycline. In one embodiment, the tetracycline compounds of the present invention inhibit autoimmune encephalomyelitis at a dosage that is approximately 90%, approximately 80%, approximately 70%, approximately 60%, approximately 50%, approximately 40%, approximately 30%, approximately 20%, or approximately 10% of the dosage of minocycline.

In one embodiment, the tetracycline compounds of the present invention, when used at the same dosage as minocycline, show better inhibition of autoimmune encephalomyelitis than minocycline. In one embodiment, the tetracycline compounds of the present invention, when used at the same dosage as minocycline, inhibit approximately 5% more, approximately 10% more, approximately 20% more, approximately 30% more, approximately 40% more, approximately 50% more, approximately 60% more, approximately 70% more, approximately 80% more, approximately 90% more, or approximately 100% more inhibition of autoimmune encephalomyelitis.

In one embodiment, the tetracycline compounds of the present invention inhibit MMP-9 and/or TNFα activity. In one embodiment, the tetracycline compounds of the present invention, when used at the same dosage as minocycline, inhibit MMP-9 and/or TNFα activity to the same extent as compared with minocycline.

In one embodiment, the tetracycline compounds of the present invention have antioxidant activity. In one embodiment, the tetracycline compounds of the present invention inhibit oxidation, such as iron-induced lipid peroxidation. In one embodiment, the tetracycline compounds of the present invention inhibit oxidation caused by oxidants, such as oxidative radicals, e.g., alkylperoxy radicals, hydrogen peroxide (H₂O₂), superoxide (O₂.⁻), hydroxyl radical (.OH), nitric oxide (NO.), peroxynitrite (ONOO⁻), and nitrosoperoxycarbonate (ONOOCO₂⁻). In one embodiment, the tetracycline compounds of the present invention inhibit oxidation at a lower concentration as compared with other tetracyclines, such as minocycline. In one embodiment, the tetracycline compounds of the present invention inhibit oxidation at a concentration at approximately or less than 100 μM, at approximately or less than 75 μM, at approximately or less than 50 μM, at approximately or less than 40 μM, at approximately or less than 30 μM, at approximately or less than 25 μM, at approximately or less than 20 μM, at approximately or less than 15 μM, at approximately or less than 10 μM, or at approximately or less than 5 μM. In a particular embodiment, the tetracycline compounds of the present invention inhibit oxidation at approximately 12.6 μM.

In one embodiment, the tetracycline compounds of the present invention display similar or improved bioavailability in the CNS as compared with other tetracycline compounds such as minocycline and doxycycline. In one embodiment, the tetracycline compounds of the present invention display similar or higher concentration in the CNS (e.g., approximately 1.1 fold, approximately 1.2 fold, approximately 1.3 fold, approximately 1.4 fold, approximately 1.5 fold, approximately 1.6 fold, approximately 1.7 fold, approximately 1.8 fold, approximately 1.9 fold, approximately 2 fold, approximately 3 fold, approximately 5 fold, approximately 6 fold, approximately 7 fold, approximately 8 fold, approximately 9 fold, approximately 10 fold, approximately 15 fold, approximately 20 fold, or approximately 30 fold) as compared to minocycline.

In one embodiment, the tetracycline compounds of the present invention have no useful anti-microbial activity and do not inhibit bacterial protein synthesis. In one embodiment, the tetracycline compounds of the present invention have a MIC value of greater than 64 μg/mL.

In one embodiment, the tetracycline compounds of the present invention display similar or improved pharmacokinetics as compared with other tetracycline compounds such as minocycline and doxycycline. In one embodiment, the tetracycline compounds of the present invention display similar or higher maximum plasma concentration (e.g., approximately 1.1 fold, approximately 1.2 fold, approximately 1.3 fold, approximately 1.4 fold, approximately 1.5 fold, approximately 1.6 fold, approximately 1.7 fold, approximately 1.8 fold, approximately 1.9 fold, approximately 2 fold, approximately 3 fold, approximately 5 fold, approximately 6 fold, approximately 7 fold, approximately 8 fold, approximately 9 fold, approximately 10 fold, approximately 15 fold, approximately 20 fold, or approximately 30 fold) as compared to minocycline. In one embodiment, the tetracycline compounds of the present invention maintains a high plasma concentration for a longer period (e.g., approximately 1.1 fold, approximately 1.2 fold, approximately 1.3 fold, approximately 1.4 fold, approximately 1.5 fold, approximately 1.6 fold, approximately 1.7 fold, approximately 1.8 fold, approximately 1.9 fold, approximately 2 fold, approximately 3 fold, approximately 5 fold, approximately 6 fold, approximately 7 fold, approximately 8 fold, approximately 9 fold, approximately 10 fold, approximately 15 fold, approximately 20 fold, or approximately 30 fold) as compared to minocycline. In one embodiment, the tetracycline compounds of the present invention reach the highest plasma concentration similar to minocycline.

The present invention also relates to a pharmaceutical composition of an effective amount of the tetracycline compounds of the present invention and a pharmaceutically acceptable carrier. The invention also relates to a pharmaceutical composition of an effective amount of a salt of the tetracycline compounds of the present invention and a pharmaceutically acceptable carrier.

The present invention also relates to a method for inhibiting, preventing, treating or ameliorating inflammation in a subject. The method includes administering to the subject an effective amount of the tetracycline compounds of the present invention or a pharmaceutical composition thereof, such that inflammation is inhibiting, prevented, treated, or ameliorated. In a specific embodiment, the tetracycline compound is Compound 1.

In an embodiment, the methods for inhibiting, preventing, treating or ameliorating inflammation as disclosed herein comprise inhibition of MMP-9 and/or TNFα activity and/or nitric oxide (NO) production by the tetracycline compounds of the present invention. In one embodiment, the tetracycline compounds of the present invention, when used at the same dosage as minocycline, inhibit MMP-9 and/or TNFα activity and/or NO production at least to the same extent as compared with minocycline. In other embodiments, the tetracycline compounds of the present invention, when used at the same dosage as minocycline, inhibit MMP-9 and/or TNFα activity and/or NO production to a greater extent than minocycline. In one embodiment, the tetracycline compound is Compound 1.

The present invention also relates to a method for treating, preventing, or ameliorating a neurodegenerative disorder (e.g., multiple sclerosis) in a subject. The method includes administering to the subject an effective amount of the tetracycline compounds of the present invention or a pharmaceutical composition thereof, such that the neurodegenerative disorder is treated, prevented, or ameliorated. In a specific embodiment, the tetracycline compound is Compound 1.

In some embodiments, the neurodegenerative disorder, e.g., multiple sclerosis, is treated with less tissue staining than caused by the same dose of minocycline. In a specific embodiment, the neurodegenerative disorder, e.g., multiple sclerosis, is treated without substantial tissue staining.

In some embodiments, the neurodegenerative disorder, e.g., multiple sclerosis, is treated with lesser antibacterial effect than caused by the same dose of minocycline. In a specific embodiment, the neurodegenerative disorder, e.g., multiple sclerosis, is treated without substantial antibacterial effect.

In an embodiment, the methods for treating, preventing, or ameliorating a neurodegenerative disorder in a subject, as disclosed herein, comprise inhibition of oxidation, e.g., lipid peroxidation and scavenging of the reactive oxygen species by the tetracycline compounds of the invention. In one embodiment, the tetracycline compounds of the present invention scavenge the reactive oxygen species, such as oxidative radicals, e.g., alkylperoxy radicals, hydrogen peroxide (H₂O₂), superoxide (O₂.⁻), hydroxyl radical (.OH), nitric oxide (NO.), peroxynitrite (ONOO⁻), and nitrosoperoxycarbonate (ONOOCO₂⁻) and inhibit oxidation caused by these species.

The methods may further comprise administering the tetracycline compounds of the present invention or a pharmaceutical composition thereof in combination with a second therapeutic agent, for example, a therapeutic agent which may enhance treatment, prevention, or amelioration of a neurodegenerative disorder (e.g., multiple sclerosis) or which may inhibit, treat, prevent or ameliorate inflammation.

The language “in combination with” a second therapeutic agent includes co-administration of the tetracycline compounds of the present invention or a pharmaceutical composition thereof and the second therapeutic agent; administration of the tetracycline compounds of the present invention or a pharmaceutical composition thereof first, followed by administration of the second therapeutic agent; and administration of the second therapeutic agent first, followed by administration of the tetracycline compounds of the present invention or a pharmaceutical composition thereof. The second therapeutic agent may be any therapeutic agent known in the art to treat, prevent, or ameliorate a neurodegenerative disorder. Furthermore, the second therapeutic agent may be any therapeutic agent of benefit to the patient when administered in combination with a tetracycline compound.

The second therapeutic agent can be any compound which treats, prevents, or ameliorates a neurodegenerative disorder. In one embodiment, the second therapeutic agent treats, prevents, or ameliorates a neurodegenerative disorder by modulating (e.g., decreasing and inhibiting) an immune response (e.g., autoimmune). In one embodiment, the second therapeutic agent treats, prevents, or ameliorates a neurodegenerative disorder by modulating (e.g., decreasing and inhibiting) inflammation. In one embodiment, the second therapeutic agent treats, prevents, or ameliorates a neurodegenerative disorder by protecting neurons or axons from damages or injuries. In one embodiment, the second therapeutic agent is a beta interferon (e.g., AVONEX® (i.e., interferon beta-1a), BETASERON® (i.e., interferon beta-1b), EXTAVIA® (i.e., interferon beta-1b), and REBIF® (i.e., interferon beta-1a)), Glatiramer (i.e., L-glutamic acid. L-alanine. L-lysin. -L-lysine copolymer (C₅H₉NO₄.C₃H₇NO₂.C₆H₁₄N₂O₂.C₉H₁₁NO₃)ₓ.xC₂H₄O₂ (e.g., COPAXONE®)), Fingolimod (i.e., 2-amino-2-[2-(4-octylphenyl)ethyl]propane-1,3-diol (e.g., GILENYA™)), Natalizumab (CAS No. 189261-10-7 (e.g., TYSABRI®)), Mitoxantrone (1,4-dihydroxy-5,8-bis[2-(2-hydroxyethylamino)ethylamino]-anthracene-9,10-dione (e.g., NOVANTRONE®)).

The term “approximate” or “approximately” means that the numeric value described herein may be 1%, 2%, 3%, 4%, 5%, 6%, 7%, 8%, 9%, or 10% higher or lower than the numeric value indicated. In one embodiment, the numeric value may be 10% higher or lower than the numeric value indicated. In one embodiment, the numeric value may be 5% higher or lower than the numeric value indicated. In one embodiment, the numeric value may be 2% higher or lower than the numeric value indicated.

The term “tetracycline compound” includes compounds with a similar tetra-fused ring structure to tetracycline. Examples of tetracycline compounds include, for example, tetracycline, oxytetracycline, sancycline, and doxycycline. For example, a tetracycline compound is the tetracycline compound of formula I, In one embodiment, the tetracycline compound is Compound 1, Compound 2 or Compound 3. In a specific embodiment, the tetracycline compound is Compound 1.

The term “alkyl” refers to a monovalent straight or branched hydrocarbon chain. Examples of straight-chain alkyl include, but are not limited to, methyl, ethyl, propyl, butyl, pentyl, hexyl, heptyl, octyl, nonyl, and decyl. Examples of branched alkyl include, but are not limited to, isopropyl, tert-butyl, and isobutyl. An alkyl group may contain 1-20 carbon atoms in its backbone for straight chain and 3-20 carbon atoms for branched chain. In one embodiment, an alkyl group may contain 1-6 carbon atoms in its backbone for straight chain and 3-6 carbon atoms for branched chain. In another embodiment, an alkyl group may contain 1-4 carbon atoms in its backbone for straight chain and 3-4 carbon atoms for branched chain.

The structures of some of the tetracycline compounds of the present invention include double bonds or asymmetric carbon atoms. Such compounds can occur as racemates, racemic mixtures, single enantiomers, individual diastereomers, diastereomeric mixtures, and cis- or trans- or E- or Z— double bond isomeric forms. Such isomers can be obtained in substantially pure form by classical separation techniques and by stereochemically controlled synthesis. Furthermore, the structures and other compounds and moieties discussed in the present invention also include all tautomers thereof.

The tetracycline compounds of the present invention may be basic or acidic, and are capable of forming a wide variety of salts with various acids or bases. The acids that may be used to prepare pharmaceutically acceptable salts of the tetracycline compounds of the present invention that are basic are those that form non-toxic acid addition salts, such as HCl salt, HBr salt, HI salt, nitrate, sulfate, bisulfate, phosphate, acid phosphate, isonicotinate, acetate, lactate, salicylate, citrate, acid citrate, tartrate, bitartrate, pantothenate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate. The bases that may be used to prepare pharmaceutically acceptable salts of the tetracycline compounds of the present invention that are acidic are those that form a non-toxic base salts, such as those salts containing alkali metal cations (e.g., Na and K), alkaline earth metal cations (e.g., Mg and Ca), and amine.

“Neurodegeneration” refers to the progressive loss of structure or function of neurons, including death or demyelination of neurons. Accordingly, a “neurodegenerative disorder” is any disorder that involves neurodegeneration. Examples of neurodegenerative disorders include, but are not limited to, Alzheimer's disease, dementias related to Alzheimer's disease (such as Pick's disease), Parkinson's disease, Lewy diffuse body diseases, senile dementia, Huntington's disease, encephalitis, Gilles de la Tourette's syndrome, multiple sclerosis, amylotropic lateral sclerosis (ALS), progressive supranuclear palsy, epilepsy, and Creutzfeldt-Jakob disease, stroke, or Fragile X syndrome. Further neurodegenerative disorders include, for example, those listed by the National Institutes of Health.

In one specific embodiment, the neurodegenerative disorder is multiple sclerosis. In another specific embodiment, the neurodegenerative disorder is Fragile X syndrome. In another specific embodiment, the neurodegenerative disorder is stroke.

In some embodiments, the neurodegenerative disorder is a disorder associated with inflammation of the brain and spinal cord, e.g., encephalomyelitis. Examples of encephalomyelitis include, but are not limited to, acute disseminated encephalomyelitis (or postinfectious encephalomyelitis); encephalomyelitis disseminate, i.e., multiple sclerosis; equine encephalomyelitis; myalgic encephalomyelitis; and autoimmune encephalomyelitis. In a specific embodiment, the neurodegenerative disorder is multiple sclerosis. In another specific embodiment, the neurodegenerative disorder is autoimmune encephalomyelitis (EAE).

In some embodiments, the neurodegenerative disorder is a demyelination associated disorder. “Demyelination” refers to damages to the myelin sheath of neurons. Demyelination can impair the conduction of signals in the affected nerves, and cause impairment in sensation, movement, cognition, or other functions depending on which nerves are involved. Demyelination is associated with many diseases in both the CNS and the peripheral nervous system, such as multiple sclerosis, Vitamin B12 deficiency, central pontine myelinolysis, Tabes Dorsalis, transverse myelitis, Devic's disease, progressive multifocal leukoencephalopathy, optic neuritis, leukodystrophies, Guillain-Barre syndrome, chronic inflammatory demyelinating polyneuropathy, anti-MAG peripheral neuropathy, Charcot-Marie-Tooth disease, and copper deficiency.

An axon, also known as a nerve fiber, is a long, slender projection of a neuron, which conducts electrical impulses. “Axon loss” or loss of axon refers to loss of structure or function of axons. Loss of axon function may be caused by damages or injuries to the axon or to the myelin sheath surrounding the axon.

The term “subject” includes humans and other animals (e.g., mammals (e.g., cats, dogs, horses, pigs, cows, sheep, rodents, rabbits, squirrels, bears, or primates)) having a neurodegenerative disorder (e.g., multiple sclerosis) or an increased risk of developing a neurodegenerative disorder (e.g., multiple sclerosis). In one embodiment, the subject is a human. In another embodiment, the subject is a mammal.

The language “effective amount” is the amount of a compound (e.g., tetracycline compound) necessary or sufficient to treat, prevent, or ameliorate a neurodegenerative disorder (e.g., multiple sclerosis) in a subject. The effective amount may vary depending on such factors as the size and weight of the subject, or the particular compound. For example, the choice of the compound may affect what constitutes an “effective amount”. One of ordinary skill in the art would be able to study the aforementioned factors and make the determination regarding the effective amount of the compound without undue experimentation.

The regimen of administration may affect what constitutes an effective amount. A compound (e.g., tetracycline compound) may be administered to the subject either prior to or after the onset of a neurodegenerative disorder (e.g., multiple sclerosis). Further, several divided dosages, as well as staggered dosages may be administered daily or sequentially; or the dose can be continuously infused, or administered orally or by inhalation, or by a bolus injection. The dosages of the compound may be proportionally increased or decreased as indicated by the exigencies of the therapeutic or prophylactic situation.

The term “treat”, “treating”, or “treatment” describes the management and care of a patient for the purpose of combating a neurodegenerative disorder (e.g., multiple sclerosis) and includes the administration of an active agent of the present invention (e.g., the tetracycline compounds or a pharmaceutical composition thereof described herein), or a pharmaceutically acceptable salt, prodrug, metabolite, polymorph or solvate thereof, to eliminate the neurodegenerative disorder.

The term “prevent”, “preventing”, or “prevention” as used herein includes either preventing the onset of a clinically evident disease progression altogether, or preventing or slowing the onset of a preclinically evident stage of a neurodegenerative disorder (e.g., multiple sclerosis) in the subject at risk. This includes prophylactic treatment of a subject at risk of suffering a neurodegenerative disorder.

The term “ameliorate”, “ameliorating”, “amelioration”, “alleviate”, “alleviating”, or “alleviation” is meant to describe a process by which the severity of a sign or symptom of a neurodegenerative disorder (e.g., multiple sclerosis) is decreased. Importantly, a sign or symptom can be ameliorated or alleviated without the neurodegenerative disorder being eliminated. In a preferred embodiment, the administration of the tetracycline compounds of the present invention or a pharmaceutical composition thereof leads to the elimination of a sign or symptom of the neurodegenerative disorder, however, elimination of the neurodegenerative disorder is not required.

The term “symptom” is defined as an indication of disease, illness, or injury, or that something is not right in the body. Symptoms are felt or noticed by the subject experiencing the symptom, but may not easily be noticed by others. Others are defined as non-health-care professionals.

The term “sign” is defined as an indication that something is not right in the body. Signs are defined as things that can be seen by a doctor, nurse, or other health care professional.

The tetracycline compounds of the invention can be synthesized by using art recognized techniques, such as those described in WO 2010/033939, WO 2005/009943, WO 2002/004406, and WO 2001/019784, the contents of each of which are incorporated herein by reference in their entirety.

The tetracycline compounds thus obtained can be further purified, for example, by flash column chromatography, high performance liquid chromatography, crystallization, or any known purification method.

The reagents used in the synthetic routes described in the above patent application publications may include, for example, solvents, reagents, catalysts, and protecting group and deprotecting group reagents. The synthetic routes may also include additional steps, either before or after the steps described specifically therein, to add or remove suitable protecting groups in order to ultimately allow synthesis of the desired tetracycline compounds. In addition, various synthetic steps may be performed in an alternate sequence or order to give the desired tetracycline compounds. For example, compounds may be further modified via conventional chemical transformations to produce compounds of the present invention. Synthetic chemistry transformations and protecting group methodologies (protection and deprotection) are known in the art and include, such as those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995).

The synthetic routes described in the above patent application publications are used only for illustrative purposes. One skilled in the art, in view of these schemes and the examples provided therein, would appreciate that all of the compounds of the present invention can be made by similar methods that are well known in the art.

### EXEMPLIFICATION OF THE INVENTION

### Example 1

A series of recent clinical studies were conducted using the tetracyclines minocycline and doxycycline for the treatment of MS. When administered at a typical antibacterial dose (200 mg/day), minocycline decreased the number of gadolinium-enhancing MRI lesions by 93%, decreased the relapse rate by 79%, and prevented worsening of disability in MS (Table 1). Mild nausea in some patients was the only adverse event noted. In additional, doxycycline in combination with IFN-β and minocycline in combination with glatiramer acetate were shown to significantly decrease lesion counts and disability scores with no increase in adverse effects. In the latter study the combination treatment was more effective than glatiramer acetate alone.

TABLE 1

| | % Relapses | % lesion | | |
|---|---|---|---|---|
| | eduction | reduction | potential side effects | administration |
| COPAXONE ® | 30 | 65 | Infection, shaking hands, | daily injection |
| | | | pain | |
| AVONEX ® | 32 | 57 | Infection, seizure liver | 3×/wk injection |
| | | | problems, pain | |
| TYSABRI ® | 68 | 83 | Liver damage, fatigue, fatal | monthly |
| | | | PML | injection |
| Fingolimod | 54 | 30 | Infections, cancer, cardiac | daily tablet |
| | | | issues | |
| Minocycline | 79 | 93 | Mild Nausea | 2×/day tablet |
| BG-12* | 32 | 69 | Flushing | 3×/day tablet |
| Laquinimod* | 21 | 40 | Increase in liver enzymes | daily tablet |
| Teriflunomid* | 30 | 44 | Mild infections, Fatigue, | daily tablet |
| | | | sensory disturbance | |

*p < 0.05

### Example 2

Compound 1 showed improved efficacy over minocycline and other approved MS therapies in accepted animal models of MS and neuroprotection (Table 2). In addition, compound 1 has a lower propensity to cause tissue staining than minocycline and has demonstrated an improved safety and pharmacokinetic profile over minocycline in pre-clinical toxicology and ADME testing.

TABLE 2

| | % Inhi- | % Inhi- | % Inhi- | % Inhi- |
|---|---|---|---|---|
| | bition | bition | bition | bition |
| | Clinical | Inflam- | Demyel- | Axon |
| Compound | Score | mation | ination | Loss |
| Fingolimod* | 89 | 59 | 69 | 60 |
| Minocycline* | 40 | 43 | 34 | 38 |
| Compound 1* | 65 | 74 | 69 | 70 |
| IFN-β | 38 | 28 | 29 | |
| Glatiramer acetate | 75 | | 74 | |

*p < 0.05

### Example 3

The inhibitory activity of Compound 1 was characterized in both mouse and rat models of EAE. Mice were immunized s.c. with myelin oligodendrocyte glycoprotein (MOG) peptide 35-55 in CFA and later injected i.v. with Pertussis toxin. Mice were randomized at day 10 and given compound i.p. Compounds were subsequently administered daily and the animals assessed for clinical score. Mice were scored as follows: 0=no disease; 1=limp tail; 2=paralysis of one or both hind limbs; 4=paralysis of both hind- and forelimbs. Lewis rats were immunized s.c. on day 1 with guinea pig myelin basic protein (MBP) emulsified in CFA. Rats were dosed daily i.p. with compound starting day 9. Rats were scored daily and cumulative scores were determined by adding the average daily scores over the experimental period.

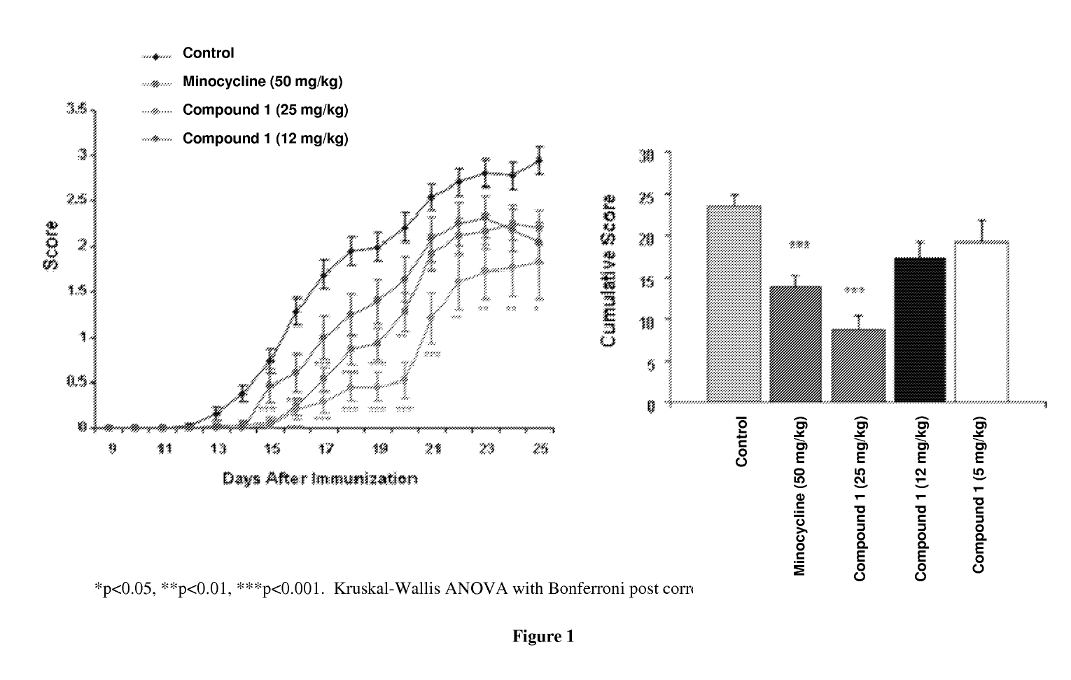

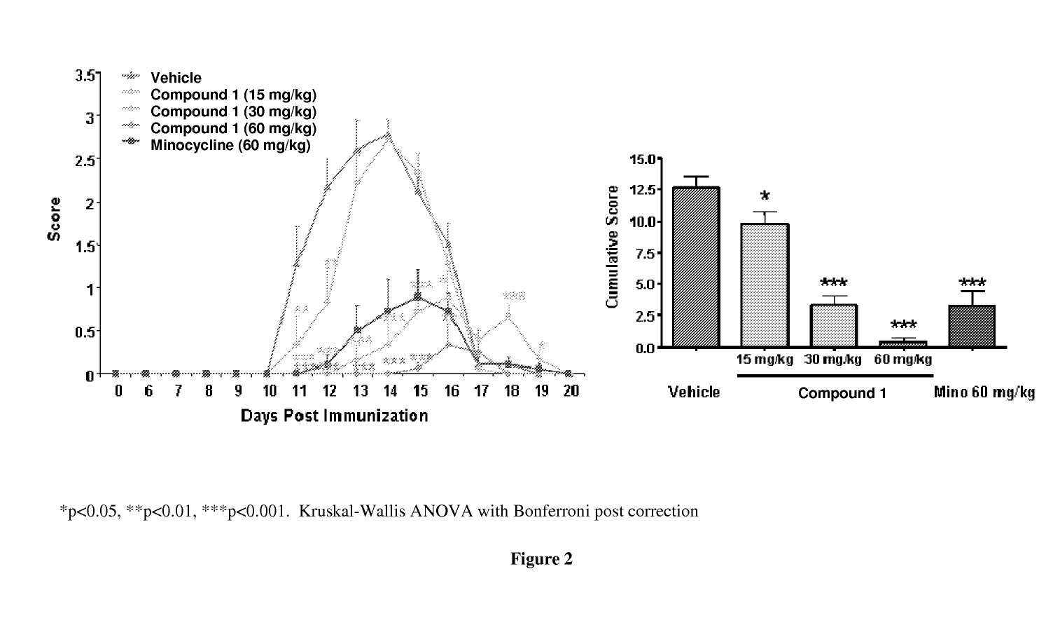

The score-dose response of EAE inhibition is shown for Compound 1 in FIG. 1 (C57BL/6 mouse model) and FIG. 2 (MBP-induced Lewis rat EAE model). The average daily scores+/−SEM and the cumulative average scores are shown. Cumulative average scores were determined by adding the average daily scores over the experimental period. Compound 1 both delayed the onset and inhibited the maximum disease severity more potently than minocycline in both animal models.

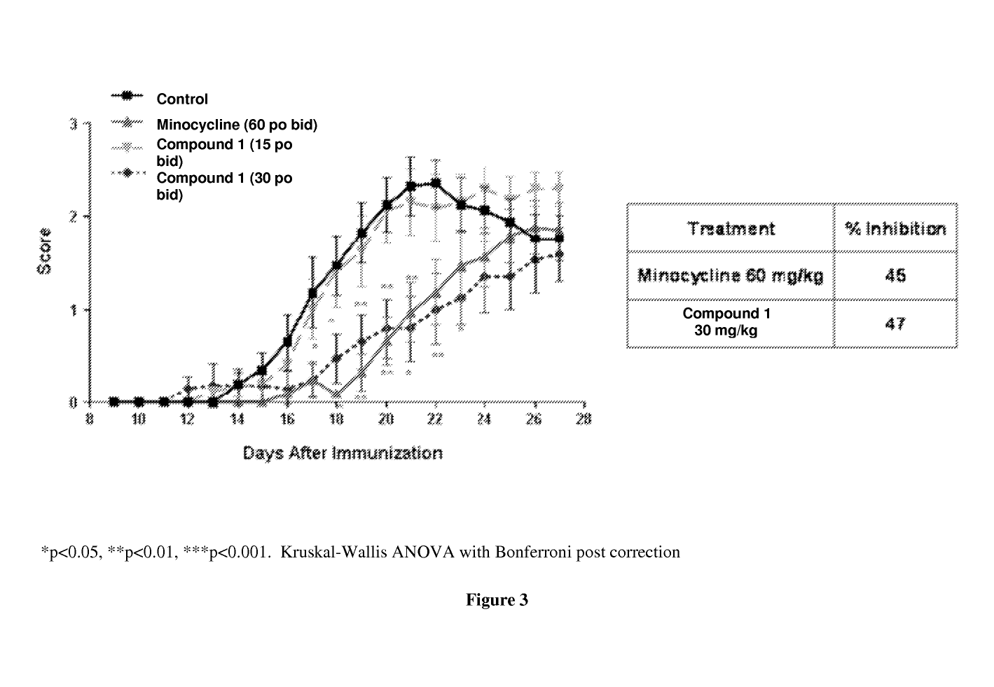

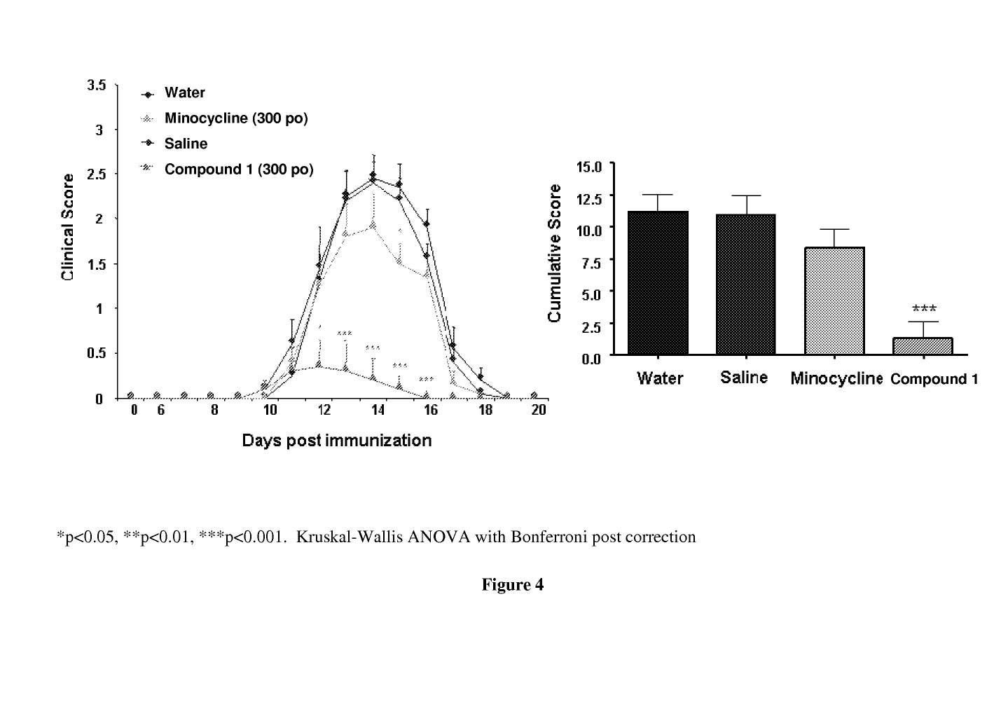

Subsequent studies to demonstrate the oral efficacy of Compound 1 were performed with experimental procedures similar to those described above, except that, in experiments leading to results in FIG. 3, compounds were administered twice daily. The results are shown in FIGS. 3 and 4.

### Example 4

Compound 1 was tested in the mouse model of cuprizone-induced demyelination to determine the protective effects of Compound 1. The general protocol for cuprizone model demyelination is as follows:

C57BL/6 female mice at 7-8 weeks old were fed a cuprizone diet (7012, 0.2% cuprizone mixed in standard pellet rodent chow purchased from Harlan Teklad, Indianapolis, Ind., USA) for 5 weeks. The cuprizone food was changed every two days and given ad libitum along with water. An additional group of mice was fed normal chow for 5 weeks to serve as a no cuprizone control.

Animals were dosed intraperitoneally (i.p.) once daily based on body weight with Compound 1 (25 mg/kg, 10 mL/kg in saline), minocycline positive control (25 mg/kg, 10 mL/kg in saline) or saline sham starting on the day of cuprizone diet initiation (day 0) and continuing until day of harvest.

At 3, 4, and 5 weeks of cuprizone feeding, ten mice from each group were euthanized by CO₂ asphyxiation and decapitation and the brains were harvested and fixed in 10% buffered neutral formalin.

Postfixed brains were paraffin embedded and 8-12 μm serial sections of the brain between the septostriatal and rostral diencephalon were prepared for luxol fast blue periodic acid-Schiff base (LFB-PAS) staining (demyelination). Both medial and lateral demyelination of the corpus callosum was determined.

Demyelination scores for the medial corpus callosum ranged from 0 to 4 (0=fully myelinated, 1=<⅓ demyelination in the center of the medial corpus callosum, 2=<⅔ demyelination in the center of the corpus callosum, 3=no myelin in the center of corpus callosum and 4=demyelination extending to the arch of the medial corpus callosum). Demyelination in the lateral corpus callosum was scored on weeks 4 and 5 and ranged from 0 to 3 (0=normal myelination and 3=complete demyelination). Demyelination was scored 2-3 times for at least 2 brain sections.

Axon loss in the medial corpus callosum was determined on additional serial sections of brain using Bielschowsky silver staining. Axon loss in the medial corpus callosum was scored from 0-4 (0=normal number of axons to 4=complete axon loss) at 3, 4 and 5 weeks. Slides were scored by two technicians who were blinded to the treatment groups. The results are shown in Table 3.

TABLE 3

| | % Inhibition | | | | | |
|---|---|---|---|---|---|---|
| | Demyelination | | | Axon Loss | | |
| Compound | Week 3 | Week 4 | Week 5 | Week 3 | Week 4 | Week 5 |
| | Medial Corpus Callosum | | | | | |
| Minocycline | 31 | 17 | 12 | 29 | 30 | −10 |
| Compound 1 | 70** | 13 | −11 | 66* | 41* | 3 |
| | Lateral Corpus Callosum | | | | | |
| Minocycline | nt | 63* | 25 | nt | nt | nt |
| Compound 1 | nt | 62* | 29 | nt | nt | nt |

nt = not tested; *p < 0.05 and **p < 0.01; Kruskal-Wallis ANOVA with Bonferroni post correction.

Compound 1 dosed i.p. at 25 mg/kg/d inhibited demyelination in the medial corpus callosum at week 3 (70% inhibition) and in the lateral corpus callosum at week 4 (62% inhibition) of cuprizone feeding. Compound 1 also significantly inhibited axon loss in the medial corpus callosum at week 3 (62% inhibition) and 4 (41% inhibition). Minocycline treatment at 25 mg/kg/d inhibited demyelination in the medial corpus callosum at 3 and 4 weeks, but the effect was not statistically significant. However, minocycline significantly inhibited lateral demyelination (63% inhibition) at 4 weeks, but had only slight effects at week 3 and 5. No statistically significant effect on axon loss was observed for minocycline treatment in this study. Most notably, Compound 1 treatment was more effective than minocycline treatment at the same dose, particularly on medial demyelination and axon loss.

### Example 5

The purpose of the study is to determine the neuroprotective effect of Compound 1.

### Materials and Methods

The method for isolation of cerebellar granule neurons has been previously described in published studies. Neonate C57BL/6 mouse pups, 7-8 days old, are obtained. Heads are removed and rinsed in 70% alcohol and transfered to an 85 mm dish filled with PBS on ice. Heads are cut, the brains removed, and the cerebella dissected and placed in a 50 mm dish with PBS on ice. The meninges, choroids plexus and blood vessels are removed from the cerebellum and the cleaned organ is transferred to a 35 mm dish with Hank's buffered salt solution (HBSS) containing Ca²⁺ and Mg²⁺. Each cerebellum is cut into pieces and incubated with trypsin solution at 37° C. for 10 minutes, after which 0.5 mg/ml (final concentration) trypsin inhibitor and 0.1 mg/ml (final concentration) DNAse are added. After centrifugation, cerebellar fragments are resuspended in dissociation medium (HBSS with Ca²⁺, Mg²⁺, trypsin inhibitor, DNAse) and a single cell suspension generated by agitation with a Pasteur pipette. After the coarse debris settles, the supernatant (cell suspension) is passed through a cell strainer (40 μm, Becton Dickinson #1942501) into a 50 ml sterile tube and the cells centrifuged. Cells are washed and re-suspended in culture medium (BME Basal Medium containing 10% fetal bovine serum, 100 IU/ml penicillin/streptomycin, 10 mM HEPES, 25 mM KCl, 2 mM L-glutamine).

The cell suspension is transferred to poly-D-lysine-coated dishes and incubated for 25 min at 37° C. in 5% CO₂. After incubation, the non-adherent cells are removed and counted with a hemocytometer. A suspension of 0.9×106 cells/ml is prepared in culture medium, and 100 μL volumes are added per well (250,000 cells/cm²) in poly-D-Lysine coated 96-well plates. One day after plating, cytosine arabinoside (AraC) is added to a final concentration of 10 μM. After 6-8 days of in vitro culture, the neurons are ready to be used in the assay.

To test the activity of Compound 1 and minocycline, compounds were added to the neuron cultures at varying concentrations. Buffer was added to some cells as a negative control. After pre-incubation with compound for 30 minutes at 5% CO₂, 37° C., glutamate was added at a final concentration of 150 mM and the cells further incubated for 1 hour. Glutamate was not added to some cells as a positive control for cell survival. After the 1 hr period, cells were washed once with culture medium and 100 μL of fresh medium were added per well. Compounds were re-added to the cells and the cultures were incubated overnight at 5% CO₂, 37° C. Following the overnight incubation, a 10 μL aliquot of a solution of 3-(4,5-dimethylthiazol-2-yl)-2,5-diphenyltetrazolium bromide (MTT) (Roche MTT Assay Kit #1465007) was added to each well and incubated for 4 hours at 5% CO₂, 37° C. Solubilization solution (100 μL/well) was subsequently added and the cells incubated overnight at 5% CO₂, 37° C. Spectrophotometric absorbance at wavelength 570 nm was measured using a microplate reader.

The MTT absorbance for non-stimulated cells was considered the 100% survival level. Glutamate-stimulated cells receiving no compound exhibited lower MTT absorbance values and their survival rate was about 50%. Increased neuron survival due to the addition of compounds was exhibited as increased MTT absorbance.

### Results

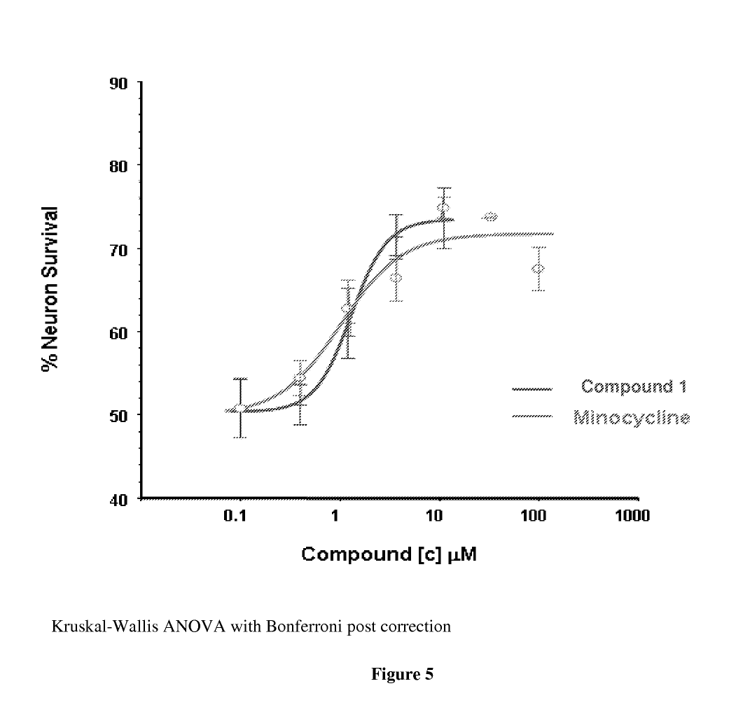

Both Compound 1 and minocycline increased the survival of glutamate-stimulated cerebellar granule neurons in a dose-dependent manner. The results are shown graphically in FIG. 5. It is evident that the efficacy of Compound 1 for inhibition of excitotoxicity is nearly identical to that of minocycline. The EC₅₀ values (the concentration at which 50% of the maximum response is observed) for the compounds are 1.4+/−0.1 μM for minocycline and 2.6 +/−0.9 μM for Compound 1.

### Example 6

Compound 1 was tested to determine specific anti-inflammatory characteristics. In vitro assays to assess the inhibition of substrate cleavage by matrix metalloproteinase 9 (MMP-9) and the inhibition of TNFα production by lipopolysaccharide (LPS)-activated macrophages were performed. MMP-9 activity was determined by fluorescence after incubation of recombinant MMP-9 with fluorescein-conjugated DQ gelatin for 90 min. TNFα production was determined by incubation of 10 ng/ml LPS with RAW264.7 macrophage cells for 24 h, followed by quantitation of TNFα in culture supernatant by ELISA.

The results are shown in Table 4. Compound 1 more potently inhibited MMP-9 enzyme activity and macrophage production of TNFα than did minocycline, and showed greater neuroprotective activity through oxygen radical scavenging.

TABLE 4

| | Neuroprotection | MMP-9 activity | TNFα production |
|---|---|---|---|
| Compound | (mouse CGN) | (cell-free) | (RAW264.7) |
| Minocycline | 1.4 ± 0.1 | 43.2 | 47 ± 4.2 |
| Compound 1 | 2.6 ± 0.9 | 29 | 10.9 ± 1.6 |

### Example 7

Tetracycline compounds were tested in an in vitro assay of ferric iron-induced lipid peroxidation among rat brain tissue. The results show that at 100 μM, Compound 1 has antioxidant activity similar to minocycline.

TABLE 5

| Concentration | Compound 1 | Minocycline |
|---|---|---|
| (μM) | (% of Control) | (% of Control) |
| 100 | 56 | 56 |
| 10 | 68 | 64 |
| 1.0 | 92 | 90 |

In a separate assay, alkylperoxy radicals were generated in vitro using the radical generator AIPH (2,2′-azobis-[2-(2-imidazolyn-2-yl)-propane) and the ability of tetracyclines to scavenge these radicals was determined. Minocycline attained an IC₅₀ value of 29 μM whereas Compound 1 was found to be 12.6 μM. Also, the ability of Compound 1 and minocycline to specifically scavenge peroxynitrite-carbonate radicals was determined. Minocycline and Compound 1 had nearly identical IC₅₀ values of 10.9 and 11.0 μM, respectively. These results also show that Compound 1 has a greater antioxidant activity than minocycline.

### Example 8

The pharmacokinetics of Compound 1 was studied in the monkey and the PK parameters, as well as more detailed data for rat pharmacokinetics, is shown in Table 6. In addition, the bioavailability of Compound 1 in the CNS of the mouse was determined and the results are shown in Table 7. Compound 1 exhibits similar PK parameters to minocycline in primates and reaches higher CNS levels in mice.

Compounds were administered via the indicated routes and blood samples were removed at various times up to 24 hr. Concentration of compound was determined by LC/MS. Cₘₐₓ=maximum plasma concentration; Tₘₐₓ=time at which Cₘₐₓ is achieved; AUC=area under the curve for 24 hrs.

TABLE 6

| Com- | Spe- | | Dose | Cₘₐₓ | Tₘₐₓ | AUC | T_{1/2} | |
|---|---|---|---|---|---|---|---|---|
| pound | cies | Route | (mg/kg) | (μM) | (hr) | (μM · hr) | (hr) | % F |
| Mino- | Rat | i.v. | 1 | 0.93 | | 2.4 | 3.6 | |
| cycline | | p.o | 5 | 0.40 | 1.7 | 3.6 | | 30.7 |
| | Mon- | i.v. | 5 | 29.9 | | 55.8 | 10.4 | |
| | key | p.o. | 5 | 4.4 | 3.0 | 44.6 | | 80.1 |
| Com- | Rat | i.v. | 5 | 27.9 | | 54.4 | 3.5 | |
| pound 1 | | p.o | 5 | 1.25 | 2.0 | 14.3 | | 26.0 |
| | Mon- | i.v. | 5 | 28.5 | | 70.0 | 5.7 | |
| | key | p.o. | 5 | 8.6 | 3.0 | 46.7 | | 66.9 |

TABLE 7

| | | | | Compound concentration (ng/ml or ng/g) | | | |
|---|---|---|---|---|---|---|---|
| Compound | Species | Dose/Route | Tissue | 0.5 hr | 1 hr | 2 hr | 4 hr |
| Minocycline | Mouse | 10 mg/kg | Plasma | 1450 | 1500 | 623 | 475 |
| | | (i.p.) | Brain | 138 | 380 | 317 | 327 |
| | | | B/P % | 9.5 | 25.3 | 50.8 | 68.8 |
| Compound 1 | Mouse | 10 mg/kg | Plasma | 6856 | 6356 | 7022 | 3678 |
| | | (i.p.) | Brain | 1227 | 1807 | 3987 | 4307 |
| | | | B/P % | 17.9 | 28.4 | 56.8 | 117.1 |

### Example 9

In the pre-clinical safety and ADME studies, Compound 1 demonstrated no significant effects in genotoxicity, CYP450 inhibition or induction, metabolism or hERG/ion channel assays. The compound was also shown to be negative in a GLP phototoxicity assay. No treatment-related effects were observed in a cardiovascular safety study in monkeys and only slight effects on fetal body weight were observed at the high dose of 250 mg/kg in a preliminary embryofetal DRF study in rats. Both 14-day acute toxicity and 28-day toxicology study were performed in rats with Compound 1 along with minocycline for comparison. Animals were administered daily oral doses from 15 to 150 mg/kg for up to 4 weeks followed by a recovery period. Few adverse effects were observed at any dose of Compound 1 in either study. Unlike minocycline, Compound 1 demonstrated little or no thyroid tissue staining at comparable doses to minocycline which demonstrates the reduced potential of the lead for causing tissue staining in patients.

### Example 10

The antibacterial activity of Compound 1 was evaluated against comparator compounds minocycline and doxycycline. Test compounds are considered to have antibacterial activity if they inhibit bacterial growth at test concentrations below 4 μM. Minocycline and doxycycline displayed strong antibacterial activity with MICs of 1.0 and 0.5 μM against E. coli, respectively (Table 8). Further, mechanistic evidence of their antibacterial activity was evaluated using the transcription/translation (TnT) in vitro assay which directly measures the bacterial cellular efficiency of protein synthesis with and without test compounds present. This TnT assay showed that both minocycline and doxycycline directly inhibit the bacterial ribosome attaining values of 1.9 and 5.4 μg/mL, respectively. However, Compound 1 has no antibacterial activity as evidenced by the >64 μM MIC value and >100 μg/mL TnT value in the above mentioned assays (Table 8).

TABLE 8

Antibacterial Activity

| | E. coli MIC | Protein Synthesis Inhibition |
|---|---|---|
| Compound | (μg/mL) | (IC₅₀ μg/mL) |
| Minocycline | 1.0 | 1.9 |
| Doxycycline | 0.5 | 5.4 |
| Compound 1 | >64 | >100 |

### Example 11

The goal of this study was to evaluate the efficacy of Compound 1 in a rat model of stroke. Male Wistar rats weighing approximately 300-350 g were used for these studies. Animals were anesthetized with chloral hydrate i.p., at 400 g/kg initially and 100 mg/kg for maintenance (for temporary occlusion model) or 5% isoflurane for induction and 1-2% for maintenance (for permanent occlusion model). Body temperature was maintained at 37° C. with a heating lamp during the operation and during the recovery period from anesthesia. After a small incision was made, local dissection was performed to expose the left femoral vein and artery. A PE-50 catheter was introduced into the left femoral vein and passed proximally to the inferior vena cava for administering drugs.

### Induction of Focal Cerebral Ischemia

The skin over the neck was shaved and a small midline incision was made. Then under the operating microscope, the right common carotid artery (CCA) was exposed. After dividing the omohyoid muscle, the CCA was isolated with a 3-0 silk suture. The external carotid artery (ECA) was also isolated and ligated with a 5-0 silk suture. Immediately after ligation of the ipsilateral proximal CCA, a 3-0 mono filament nylon suture (occluder), its tip rounded by flame heat, was introduced into the right CCA lumen through a small incision. The occluder was gently advanced into the ICA from the CCA bifurcation. This allowed the tip of the occluder to reach the proximal portion of the anterior cerebral artery (ACA) and occlude the origin of the MCA and the PcomA. The occluder was fixed within the CCA by double ligations using a 3-0 silk suture. In temporary occlusion animals, recirculation was performed by pulling the occluder out of the ICA at 90 min after MCA occlusion.

### Administration of Compound 1

Compound 1 was dissolved in normal saline. Rats received Compound 1 treatment through femoral vein infusion at various time points before or after MCA occlusion and at concentrations of 20, 25, or 40 mg/kg. Control animals were treated with an equal volume of saline.

### Neurological Evaluation

Neurological deficit was evaluated at 4 and 24 hours after MCA occlusion according to a six-point scale: 0=no neurological deficits, 1=failure to extend left forepaw fully, 2=circling to the left, 3=falling to left, 4=no spontaneous walking with a depressed level of consciousness, and 5=death (Candelario-Jalil E, et al, Brain Research, 2004; Hattori K, et al, Stroke, 2000; Gerriets T, et al, stroke, 2004). Any animal with a neurological finding of greater than 4 was humanely euthanized.

### Assessment of Brain Infarct Volume

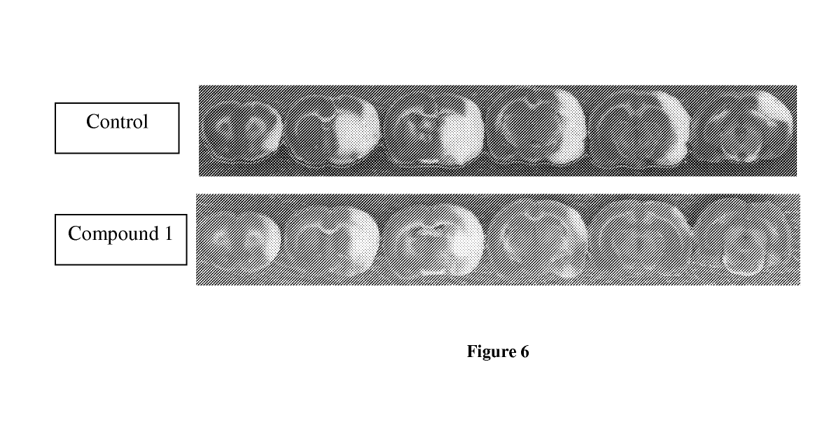

After completing the neurological evaluation at 24 hr after MCAO, the animals were sacrificed through an overdose of anesthesia and brains were removed, frozen, and coronally sectioned into six 2-mm-thick slices. The brain slices then were incubated at 37° C. for 30 min in a 2% solution of 2,3,5-triphenyltetrazolium chloride (TTC) and fixed by immersion in a 10% formalin. TTC-stained brain sections were digitized using a color flatbed scanner and analyzed using image processing software. A corrected infarct volume was calculated to compensate for the effect of brain edema. Presented in FIG. 6 are sample brain slices from 90 min temporary MCA occluded rats stained with TTC. The red areas represent normal tissues and the white areas are infarctions.

### Statistical Analysis

Data are presented as means±SD. Statistical comparisons between drug-treated and control groups were made using a student's t test. P<0.05 was considered statistical significance.

### Results

Compound 1 showed neuroprotective effects in the 90 min temporary occlusion model with treatment started at 30 or 60 min after MCA occlusion and in the permanent occlusion model with the treatment started at 90 min pre-occlusion, statistically significant reductions in infarct volume were noted. However, Compound 1 did not demonstrate an ability to reduce infarct volume when treatment was initiated three hours post occlusion in temporary occluded rats.

TABLE 9
Effects of Compound 1 in temporary and permanent rat stroke models.
Statistically significant changes from control are bolded.

| Compound | Treat- | Num- | | | |
|---|---|---|---|---|---|
| 1 Concen- | ments (at | ber of | Infarct | Neuro- | |
| tration | time after | ani- | volume (mm3, | logical | |
| (mg/kg) | MCAO) | mals | Means ± SD) | score | model |
| 20 | 30 min, | 8 | 139.2 ± 32.2 | 1.5 ± 0.5 | tempo- |
| | 6 hr | | (174.6 ± 31.9)* | (1.7 ± 0.6) | rary |
| 40 | 60 min, | 10 | 125.9 ± 49.9 | 1.3 ± 0.5 | tempo- |
| | 6 hr | | (174.6 ± 31.9) | (1.7 ± 0.6) | rary |
| 40 | 3 hr, 8 hr | 20 | 172.8 ± 45.3 | 1.4 ± 0.5 | tempo- |
| | | | (174.6 ± 31.9) | (1.7 ± 0.6) | rary |
| 40 | −90 min, | 10 | 145.7 ± 33.9 | 1.1 ± 0.3 | perma- |
| | 90 min, | | (188.4 ± 30.8) | (1.3 ± 0.5) | nent |
| | 6 hr | | | | |

*Data in parenthesis ( ) is the data from control animals (temporary groups, n = 21; permanent groups, n = 10)

### Conclusions

Compound 1 showed neuroprotective effects by reducing the infarct volume in temporary and permanent occlusion rat stroke models. Compounds with high potency are needed to explore the neuroprotective effects of TCs in permanent occlusion model and extend the therapeutic time window in temporary occlusion models.

### Example 12

Inflammatory conditions are characterized by increasing concentrations of reactive oxygen species. In this study, the ability of Compound 1 to specifically scavenge peroxynitrite-carbonate radicals was determined.

For 1×96-well plate assay, the following volumes of solutions were prepared:

| Solution | Concentration | | Volume (mL) | Storage |
|---|---|---|---|---|
| PC buffer | 75 mM phosphate, | | 20 | Prepare fresh |
| sodium hydrogen | 25 mM sodium | | | daily |
| carbonate (MW 84.01) | hydrogen carbonate | | | |
| 1M phosphate buffer | | | | |
| DHR Stock (DS) | 1 | mM | 1 | −80° C., 2 |
| dihydrorhodamine 123 | | | | freeze-thaw |
| (MW 346.4) | | | | cycles only |
| DHR Test Compound Prep | 100 | μM | 1 | Ice, prepare |
| Solution (DTCP) | | | | fresh daily |
| DHR Working Solution (DWS) | 12.5 | μM | 10 | Ice, prepare |
| | | | | fresh daily |
| Test Compound Stock (TCS) | 5-20 | mM | Approx 1 - | Ice, store |
| | | | enough for 10-40 | aliquots |
| | | | TCWSs at | at −80° C. |
| | | | 1.25 mM | |
| Test Compound Working | 1.25x highest | | 0.4 for 2 IC₅₀ | Ice, prepare |
| Solution (TCWS) | assay concentration | | runs in 24 | fresh daily |
| | | | assay wells | |
| Sin-1 Working Solution (Sin-1) | 2.5 | mM | 5 | Ice, prepare |
| Sin-1 chloride (3- | | | | fresh prior to |
| morpholino-sydnonimine, | | | | assay addition |
| MW 207) | | | | |

| | | | |
|---|---|---|---|
| Ascorbic acid (AA) | 1M | 7 | Ice, prepare |
| ascorbic acid (MW 176) | | | fresh daily |

### Plate Prep

To the first column of a 96-well PCR plate (0.2 mL volume) was added 160 μL of TCWS. One well of the first column would equal one IC₅₀ determination. Typically, test compounds were assayed in duplicate, i.e. two wells of column 1 were filled with each TCWS. To the remaining wells in columns 2-11 was added 80 μL of DWS. Using a multichannel pipettor, column 1 was serially diluted in 2-fold dilutions by removing 80 μL from column 1 and transferring with mixing to column 2. Column 2 was then diluted to column 3 and so on until column 11 where 80 μL of the diluted mixture was removed and discarded. At least 2 wells of row 12 were designated as the 0 test compound control to which 80 μL of DWS was added. One well of row 12 per test compound was designated as the background control to which 80 μL of the TCWS was added.

### Assay

The plate was covered and incubated for 5 minutes at 37° C. upon which the reaction was initiated by addition of 20 μL of Sin-1 to columns 1-11 using a multichannel pipettor with mixing. The reaction was initiated similarly to the 0 test compound wells of column 12. To the background control wells was added 20 μL of PC buffer. The plate was incubated at 37° C. for 8 minutes (the reaction is linear for 10 minutes) and quenched by addition of 50 μL of AA to all wells with mixing. The plate was placed on ice for 5 minutes. Quenched assay mixtures were stable at room temperature for at least 24 hours.

### HPLC Analysis

Each reaction mixture (20 μL) was analyzed for rhodamine 123 product by HPLC using a Phenomenex Luna C18(2) column, 3 um, 4.6×50 mm using an A buffer of water+0.1% TFA and a B buffer of acetonitrile+0.1% TFA and the following gradient method:

| Time | | Flow rate |
|---|---|---|
| (min) | B % | (mL/min) |
| 0 | 20 | 1.5 |
| 5 | 40 | 1.5 |
| 5.1 | 100 | 1.5 |
| 6.1 | 100 | 1.5 |
| 6.2 | 20 | 1.5 |
| 8 | 20 | 1.5 |

Product rhodamine 123 was detected by UV-vis at 500 nm with a typical retention time of 4.0 minutes and the AUC was determined by integration (the retention time and AUC linearity of rhodamine 123 was established by injections of authentic rhodamine 123 at various concentrations). The absorbance at 280 nm was also recorded to ensure no co-elution of test compound peaks.

### Calculations

Initial examination of the background control samples at the highest test compound concentration were performed to ensure no co-elution of test compound peaks with the product rhodamine 123 at 4 minutes. If co-elution was observed, the compound was not tested until an appropriate HPLC method was developed to separate the product and test compound peaks. The rhodamine 123 AUC was then determined for each test compound concentration and the percent inhibition of rhodamine 123 fluorescence was then determined by the following equation:

% Inhibition=[(AUCₒ−AUCₓ)/AUCₒ]*100

Where AUCₒ is the AUC of the rhodamine 123 peak at 0 test compound concentration and AUC_{X} is the AUC of the rhodamine 123 peak at each test compound concentration=x.

The IC₅₀ (concentration at which test compound inhibits the oxidation of dihydrorhodamine 123 to rhodamine 123 by 50%) was determined from the plot of % Inhibition versus concentration using a 4-parameter logistic or sigmoidal dose response model. The standard error of the curve fit for the IC₅₀ was also determined along with the Hill slope. The IC₅₀ determined for uric acid was divided by the IC₅₀ determined for each test compound to generate Uric Acid Equivalents, a measure of the peroxynitrite-carbonate radical scavenging ability relative to uric acid.

Uric Acid Equivalents=IC₅₀ Test Compound/IC₅₀ Uric Acid

### Results

A summary of the assay results can be seen below in Table 10. Compared to uric acid, minocycline was equivalent in its ability to scavenge peroxynitrite-carbonate radicals in this assay. Other commercially-available tetracyclines were not as effective as minocycline with the exception of methacycline which was approximately 3-fold more effective in uric acid equivalents. Compound 1 demonstrated activity at least as good as minocycline

TABLE 10
Results of peroxynitrite-carbonate scavenging assay.
IC₅₀ is the compound concentration required to inhibit oxidation
of DHR probe by 50%. IC₅₀ SE is the standard error of the
IC₅₀ from the curve fit.

| | | | | | Uric Acid |
|---|---|---|---|---|---|
| Compound | n | IC₅₀ μM | IC₅₀ μM SE | Hill Slope | Equivalents |
| Uric acid | 3 | 12.4 | 0.81 | 1.6 | 1 |
| Minocycline | 3 | 10.9 | 0.79 | 1.8 | 1.14 |
| Doxycycline | 3 | 25.0 | 2.87 | 0.7 | 0.50 |
| Methacycline | 2 | 4.1 | 0.12 | 0.8 | 3.01 |
| sancycline | 2 | 66.4 | 6.21 | 1.4 | 0.19 |
| Oxytetracycline | 2 | 132.6 | 6.50 | 1.4 | 0.09 |
| Compound 1 | 4 | 11.0 | 1.18 | 1.7 | 1.13 |

### Example 13

The purpose of the study was to determine the anti-inflammatory activities of Compound 1. Minocycline was tested as a comparator compound.

### Materials and Methods

### MMP-9 Enzyme Activity Assay

This assay was designed to measure the degradation of substrate by purified enzyme. To a solution containing 2.5 μg/ml fluorescein-conjugated DQ gelatin (Invitrogen) in buffer (50 mM Tris-HCl, 150 mM NaCl, 5 mM CaCl₂, 0.2 mM sodium azide, pH 7.6), tetracycline compounds were added at final concentrations ranging from 100 to 1 μM. Subsequently, an aliquot of active recombinant human matrix metalloproteinase-9 (MMP-9)(CalBioChem) was added to a final concentration of 0.05 μg/ml. The total volume of the reaction mixture was 200 μL and samples were contained in 96-well black plates (Corning). The mixture was incubated at room temperature in the dark for 85 min, after which the fluorescence was measured using a microplate reader. Samples containing no MMP-9 enzyme were used as negative controls and samples with enzyme and without compound were positive controls.

### NO Production Assay

The J774A.1 mouse macrophage cell line was grown to confluence in DMEM medium containing 10% fetal bovine serum (FBS). Cells were harvested into single-cell suspensions (by incubation on ice and agitation), seeded into 96-well plates at 1×10⁵ cells/well (200 μL volume) and incubated (5% CO₂, 37° C.) overnight. Compounds were added to the cells at final concentrations ranging from 50 to 1 μM and pre-incubated for 1 hr. Lipopolysaccharide (LPS) was added to the cells at a final concentration of 10 ng/ml. After incubation for 20 hr, culture supernatants were harvested and transferred to a new 96-well plate. Levels of LPS-induced NO in the supernatants were quantified by Greiss reagent (Active Motif) with supernatants from unstimulated cells serving as a negative control.

### TNFα Production Assay

The RAW 264.7 mouse macrophage cell line was grown to confluence in DMEM medium containing 10% fetal bovine serum (FBS). Cells were harvested into single-cell suspensions, seeded into 96-well plates at 1-2×10⁵ cells/well (200 μL volume) and incubated (5% CO₂, 37° C.) overnight. Compounds were added to the cells at final concentrations ranging from 50 to 1 μM and pre-incubated for 30 min. Lipopolysaccharide (LPS) was added to the cells at a final concentration of 10 ng/ml. After incubation for 20 hr, culture supernatants were harvested and transferred to a new 96-well plate. Levels of LPS-induced TNFα in the supernatants were quantified by ELISA (R & D Systems) with supernatants from unstimulated cells serving as a negative control.

### Results

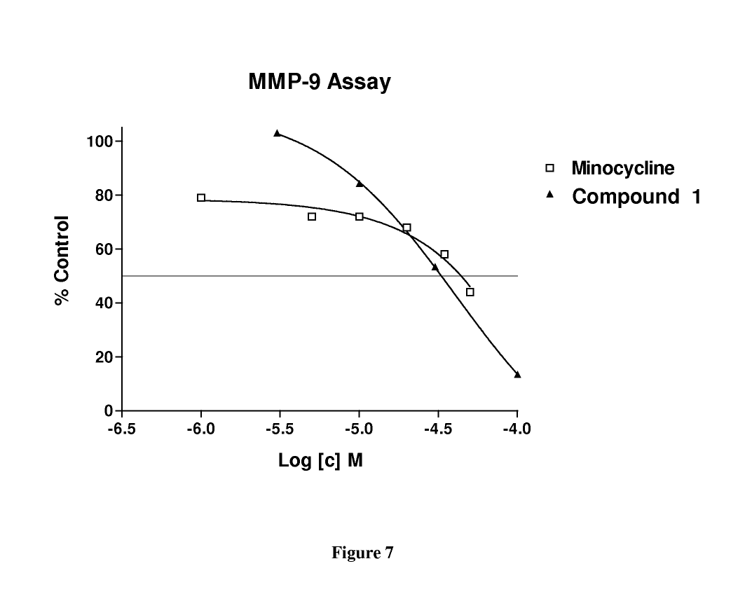

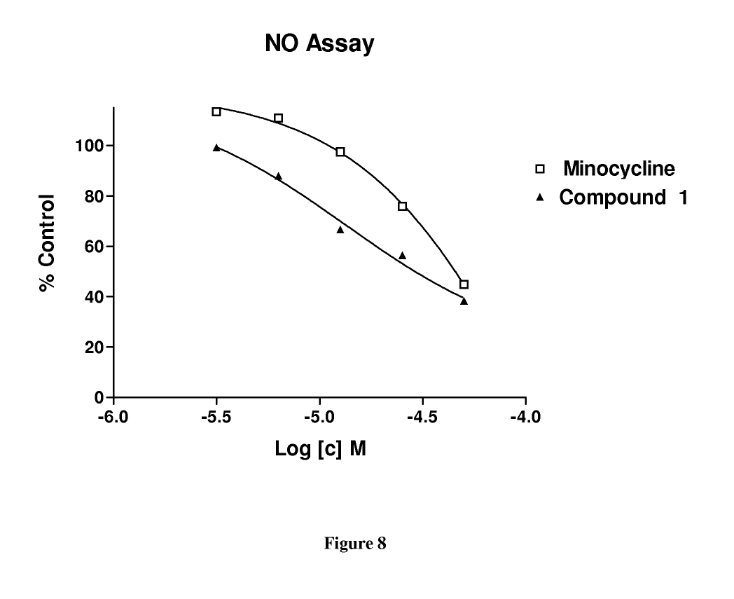

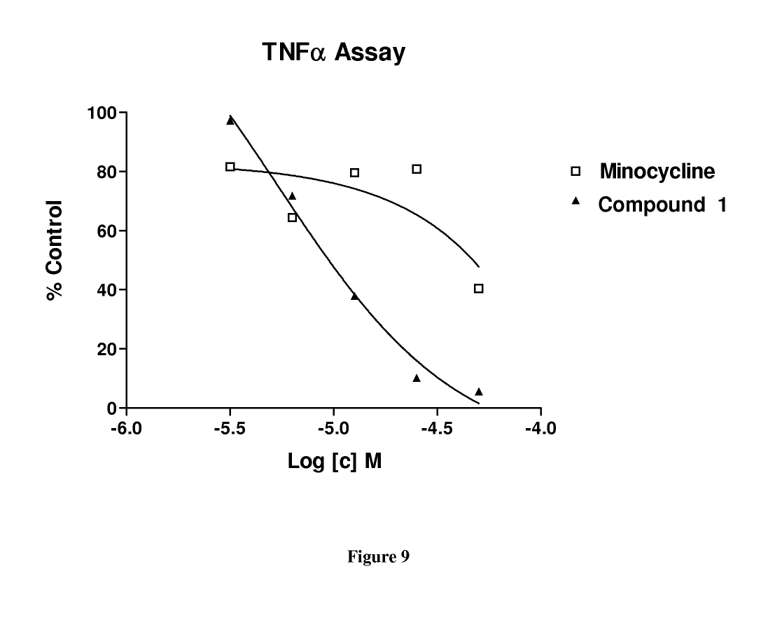

The dose responses for minocycline and Compound 1 in the in vitro MMP-9 enzyme assay, NO production assay, and TNF□ production assays are shown in FIGS. 7, 8 and 9 respectively. The IC₅₀ for the two compounds in the assays are summarized in Table 11. Though both tetracyclines exhibit inhibitory activity in these assays, Compound 1 is more potent than minocycline.

TABLE 11
IC₅₀s of minocycline and Compound 1 in the in vitro assays.

| | MMP-9 Activity | NO Production | TNFα Production |
|---|---|---|---|
| | (cell-free) | (J774A.1) | (RAW264.7) |
| Compound | IC₅₀ μM | IC₅₀ μM | IC₅₀ μM |
| Minocycline | 43.2 | 46 ± 2.1 | 47 ± 4.2 |
| Compound | 32.7 | 30 | 10.9 ± 1.6 |
| 1 | | | |

### Example 14

In this study, alkylperoxy radicals were generated in vitro using the radical generator AIPH (2,2′-azobis-[2-(2-imidazolyn-2-yl)-propane) and the ability of tetracyclines, such as Compound 1, to scavenge these radicals was determined.

### Assay Principle

Trolox is a known scavenger of peroxyradicals. Structurally similar to a-tocopherol, Trolox reacts with peroxyradicals via a known mechanism with linear kinetics under excess oxygen conditions. In the presence of a competing antioxidant compound, the rate of Trolox oxidation will change based on the relative rate of oxidation and concentration of the competing antioxidant compound (Huang, et al., J. Agric. Food Chem. 2005, 25, 1841-1856). By measuring the effect of antioxidant concentration on the rate of degradation of Trolox, the relative antioxidant capacity of a compound can be determined. This principle is the basis of the widely used antioxidant capacity assay, ORAC (Huang, et al., J. Agric. Food Chem. 2002, 50, 1815-1821). Unlike ORAC which uses a fluorescent dye indicator, the assay described here directly measures the oxidation of the Trolox derivative, 2-aminoethyl-Trolox (AE-Trolox). This method eliminates interference of the fluorescent dye reaction by tetracycline compounds.

### Materials

### Phosphate Buffer (PB Buffer)

All assays were performed in Phosphate buffer (PB buffer) (1M sodium phosphate buffer diluted to 75 mM with water and adjusted to pH 7.5 with HCl).

### 6-Hydroxy-2,5,7,8-tetramethyl-chroman-2-carboxylic acid (2-amino-ethyl)-amide (Alkylperoxyradical probe)(AE-Trolox)

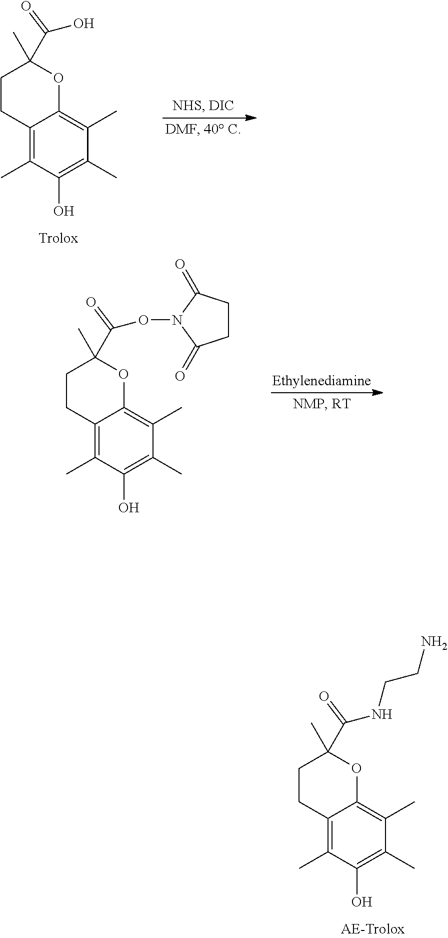 [SMILES: Cc1c(C)c2c(c(C)c1O)CCC(C)(C(=O)O)O2 ; Cc1c(C)c2c(c(C)c1O)CCC(C)(C(=O)ON1C(=O)CCC1=O)O2 ; Cc1c(C)c2c(c(C)c1O)CCC(C)(C(=O)NCCN)O2]

Synthesis:

To 30 mL of DMF was added 3.4 g of Trolox and 1.9 g of N-hydroxysuccinimide and solution was heated to 40° C. With stirring, 2.52 g (2.52 mL) of diisopropylcarbodiimide was added. After 30 minutes, the reaction was complete. The reaction was diluted to 200 mL with ethyl acetate and the organic layer was washed 3 times with 200 mL aliquots of water, then 100 mL of saturated sodium chloride in water. The organic layer was collected and dried of magnesium sulfate and evaporated to dryness to yield 4.3 g of a light beige powder. To 20 mL of NMP, 1.35 g of NHS-Trolox was added and the solution was added rapidly to 0.5 g of ethylene diamine dissolved in 20 mL of NMP at room temperature. After 10 minutes, the reaction was complete. The reaction was diluted to 1 liter with water and the pH was adjusted to 2 with TFA. The solution was filtered and the product was purified by prep-HPLC using TFA buffer and acetonitrile. Pure fractions were loaded onto RP column and washed with 3 equivalents of HCl using 0.1% HCl solution in water. Pure HCl salt was eluted with acetonitrile and evaporated to dryness to yield AE-Trolox HCl.

Stock solutions of AE-Trolox were prepared in water at 10 mM and serially diluted with PB buffer to 0.1 mM. AE-Trolox working solution (AET-WS) was prepared by diluting the 0.1 mM stock (625 μL) with 9.375 mL of PB buffer.

### 6-Hydroxy-2,5,7,8-tetramethyl-chroman-2-carboxylic acid (2-amino-ethyl-d4)-amide (Internal standard for probe, AE-Trolox-d4)

The stable isotope-labeled internal standard of AE-Trolox was prepared exactly as AE-Trolox except ethylene-d4-diamine was substituted as the reagent in the final step. Stock solutions of AE-Trolox-d4 were prepared at 100 μM in water.

### Test Compounds

Test compound stock solutions (TCS) were prepared initially in water at 5-20 mM depending on solubility. In some cases, small volumes of 6N HCl or 10N NaOH were added to achieve solubility. Test compound working solutions (TCWS) were prepared by combining appropriate volumes of test compound stock, 0.1 mM AE-Trolox stock solution and PB buffer to achieve a test compound concentration of 1.25× the highest desired assay concentration and AE-Trolox concentration of 6.25 μM. The test compound working solution was kept on ice.

### 2,2′-azobis-[2-(2-imidazolyn-2-yl)-propane (AIPH)

Just prior to assay, the AIPH solution was prepared by dissolving 80.75 mg of AIPH in 10 mL of PB buffer (25 mM AIPH). The solution was kept on ice.

Ascorbic Acid Quench Solution with Internal Standard (AA)

Ascorbic acid quench solution (approximately 6 mL) was prepared by dissolving solid ascorbic acid (1056 mg) in water to a final concentration of 1M. To this solution was added 375 μL of 0.1 mM AE-Trolox-d4 stock solution.

Reagent Amounts—96 assays
For 1×96-well plate assay, the following volumes of solutions were prepared:

| | | | Volume | |
|---|---|---|---|---|
| Solution | Concentration | | (mL) | Storage |
| PB buffer | 75 mM phosphate, | | 40 | Room temp |
| 1M phosphate buffer | pH 7.5 | | | |
| AE-Trolox Stock | 100 | μM | 1 | −80° C., 2 freeze- |
| AE-Trolox HCl (MW 328.5) | | | | thaw cycles only |
| AE-Trolox Working Solution (AET- | 6.25 | μM | 10 | Ice, prepare fresh |
| WS) | | | | daily |
| AE-Trolox-d4 Stock (AE-Trolox-d4) | 100M | | 0.5 | Ice, prepare fresh |
| AE-Trolox-d4 HCl (MW 332.5) | | | | daily |
| Test Compound Stock (TCS) | 5-20 | mM | Approx 1 - | Ice, store aliquots |
| | | | enough for | at −80° C. |
| | | | 10-40 | |
| | | | TCWSs at | |
| | | | 1.25 mM | |
| Test Compound Working Solution | 1.25x highest assay | | 0.4 for 2 | Ice, prepare fresh |
| (TCWS) | concentration, 6.25 | | IC₅₀ runs in | daily |
| | μM AE-Trolox | | 24 assay | |
| | | | wells | |
| AIPH | 25 | mM | 10 | Ice, prepare fresh |
| 2,2′-azobis-[2-(2-imidazolyn-2- | | | | prior to assay |
| yl)-propane MW 323 | | | | addition |

| | | | |
|---|---|---|---|
| Ascorbic acid (AA) | 1M AA, 6.25 μM | 6 | Ice, prepare fresh |
| ascorbic acid (MW 176) | AE-Trolox-d4 | | daily |
| AE-Trolox-d4 Stock - 100 μM | | | |

### Methods

### Plate Prep

To the first column of a 96-well PCR plate (0.2 mL volume) was added 160 μL of TCWS. One well of the first column would equal one IC₅₀ determination. Typically, test compounds were assayed in duplicate, i.e. two wells of column 1 were filled with each TCWS. To the remaining wells in columns 2-11 was added 80 μL of AET-WS. Using a multichannel pipettor, column 1 was serially diluted in 2-fold dilutions by removing 80 μL from column 1 and transferring with mixing to column 2. Column 2 was then diluted to column 3 and so on until column 11 where 80 μL of the diluted mixture was removed and discarded. Four wells of row 12 were designated as the 0 test compound/0 AIPH control to which 80 μL of AET-WS was added. One well of row 12 per test compound was designated as the 0 test compound control to which 80 μL of the AET-WS was added.

### Assay

The plate was covered and incubated for 5 minutes at 37° C. upon which the reaction was initiated by addition of 20 μL of AIPH to columns 1-11 using a multichannel pipettor with mixing. The reaction was initiated similarly to the 0 test compound wells of column 12. To the 0 test compound/0 AIPH control wells was added 20 μL of PB buffer. The plate was incubated at 37° C. for 8 minutes (the reaction is linear for 10 minutes) and quenched by addition of 50 μL of AA to all wells with mixing. The plate was placed on ice for 5 minutes. Quenched assay mixtures were stable at room temperature for at least 24 hours.

### 2D-LCMS Analysis

Each reaction mixture was analyzed for AE-Trolox by 2D-LCMS on a Shimadzu 2010 equipped with a loading column (Shim-pack MAYI-ODS, 4.6×10 mm) and a gradient column Phenomenex Luna C18(2) column, 3 um, 4.6×50 mm. Using an automated 2-position swithching valve, samples were loaded onto the loading column by Pump C and washed for 1 minute with loading buffer. At 1 minute, the valve was switched to elution position (Position B, reversed flow through loading column) and the samples were eluted through the gradient column. The loading buffer (Buffer C) was 10% acetonitrile in water with 0.2% formic acid and the gradient buffers were water+0.2% formic acid (A buffer) and acetonitrile+0.2% formic acid (B buffer). The loading and elution gradient is shown in Table 1. Typical injection volumes were 30 μL.

The MS was set to positive ion SIM mode for ions at MH+=293 (AE-Trolox) and 297 (AE-Trolox-d4). The detector voltage was 1.5 kV, CDL and Block temperature were 250° C. and the nebulizer gas was set at 5 L/min.

### 2D-LCMS Method

| Time | | A + B Flow Rate | C Flow rate | Valve |
|---|---|---|---|---|
| (min) | B % | (mL/min) | (mL/min) | Position |
| 0 | 1 | 1 | 3 | A |
| 1 | 1 | 1 | 3 | A |
| 1 | 1 | 1 | 0.2 | B |
| 6 | 100 | 1 | 0.2 | B |
| 8 | 100 | 1 | | B |
| 8.1 | 1 | 1 | | B |
| 10 | 1 | 1 | | B |

AE-Trolox and AE-Trolox-d4 co-eluted at a retention time of 3.34 minutes and the AUC of each ion from the TIC was determined by integration. The linearity of AE-Trolox AUC was determined from 100-0.14 pmol.

### Calculations

From the TIC integration of AE-Trolox and AE-Trolox-d4, a ratio was calculated:

Ratio=AUC_{AE-Trolox}/AUC_{AE-Trolox-d4}

Percent inhibition of AE-Trolox oxidation (measured as disappearance of AE-Trolox) was then determined by:

% Inhibition=[1−((Ratio₀−Ratioₓ)/(Ratioₒ−Ratio_{f}))]*100

Where Ratioₒ is the average AUC ratio of the 0 test compound/0 AIPH control assays, Ratioₓ is the AUC ratio of test compound assays at various concentrations and Ratio_{f} is the average AUC ratio of the 0 test compound control assays. Typically, Ratio₀ and Ratio_{f} where determined by averaging the 4 control wells for each.

The IC₅₀ (concentration at which test compound inhibits the oxidation of AE-Trolox by 50%) was determined from the plot of % Inhibition versus concentration using a 4-parameter logistic or sigmoidal dose response model. The standard error of the curve fit for the IC₅₀ was also determined along with the Hill slope. The IC₅₀ determined for Trolox was divided by the IC₅₀ determined for each test compound to generate Trolox Equivalents, a measure of the alkylperoxy radical scavenging ability relative to Trolox.

Trolox Equivalents=IC₅₀ Test Compound/IC₅₀ Trolox

### Results

### Time Course of AE-Trolox Oxidation

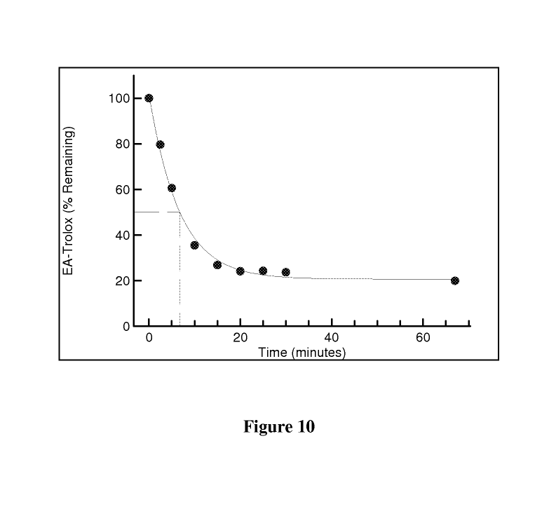

The time course of AE-Trolox oxidation (5 μM) was carried out at 5 mM AIPH. A concentration of 5 mM AIPH was chosen since it generates a radical flux rate of 26 nmol/s of alkylperoxy radical. This maintained an excess of oxygen (approximately 10-fold) over the course of 10 minutes. As seen in FIG. 10, the disappearance of AE-Trolox was linear over 10 minutes after which the rate dramatically slowed.

### IC₅₀ Determinations

Each test compound was assayed at least 3 times and all data points were combined to generate one IC₅₀ curve. The IC₅₀ data is shown in the Table 12 below.

TABLE 12
Summary of IC₅₀ determinations.

| | | | | | Trolox | Percent |
|---|---|---|---|---|---|---|
| | | | | | Equiv- | Trolox |
| Compound | n | IC₅₀ (μM) | IC₅₀ SE | h | alents | Activity |
| Trolox | 5 | 9.0 | 0.43 | 2.2 | 1.00 | 100 |
| Minocycline | 5 | 29.4 | 2.14 | 1.0 | 0.31 | 31 |
| Sancycline | 3 | 368.9 | 21.38 | 2.4 | 0.02 | 2 |
| Methacycline | 3 | 135.6 | 18.26 | 1.7 | 0.07 | 7 |
| Oxytetracycline | 3 | 1353.9 | 180.15 | 1.3 | 0.01 | 1 |
| Chlortetracycline | 3 | 61.6 | 8.98 | 0.9 | 0.15 | 15 |
| Doxycycline | 4 | 25.0 | 1.31 | 2.8 | 0.36 | 36 |
| Compound 1 | 4 | 12.6 | 0.90 | 1.0 | 0.71 | 71 |

IC₅₀ SE indicates standard error of IC₅₀ curve fit; h indicates Hill slope.

### Conclusions

Compound 1 was better than minocycline and other tetracycline analogs at scavenging alkylperoxy radicals.

### Example 15

The purpose of this study was to test Compound 1 for treating Fragile X Syndrome. This study utilized Fmr1 KO mice, an animal model of Fragile X Syndrome. The Fmr1 KO mice were tested, along with their wild-type litter mate control mice, on a range of behavior paradigms with previously and newly demonstrated efficacy in detecting the most robust phenotypic differences suited for preclinical therapeutic efficacy studies in the Fmr1 KO mutant mouse. Behavioral tests that were found to robustly discriminate Fmr1 KO mice from their wild-type littermates were used in the studies.

### Materials and Methods

The Fmr1 KO mice (C57BL/6 background) were kindly provided the FRAXA Foundation. Mice were housed in groups of the same genotype in a temperature and humidity controlled room with a 12-h light-dark cycle (lights on 7 am to 7 pm). Testing was conducted during the light phase. Food and water were available ad libitum. Testing was conducted on Fmr1 KO mice and their wild-type littermates. They were housed in commercial plastic cages purchased in the UK. Experiments were conducted in line with the requirements of the UK Animals (Scientific Procedures) Act, 1986.

The dosing protocol used is shown below. Mice were dosed at 6-7 weeks by intraperitoneal (i.p.) infusion by osmotic pump (0.5 μl/hour) for 1 week. The solution of 0.9% saline was used as a vehicle, and 10 mice were dosed per each group.

| | | | Dose Soln |
|---|---|---|---|
| | | Dose | Conc |
| Group | Strain | (mg/kg/d) | (mg/mL) |
| WT Control | WT (C57BL/6) | — | — |
| Disease Control | Fmr1 KO (C57BL/6 bckgnd) | — | — |
| Minocycline WT | Fmr1 KO WT littermates | 10 | 18.3† |
| control | (C57BL/6 bckgnd) | | |
| Minocycline | Fmr1 KO (C57BL/6 bckgnd) | 10 | 18.3† |
| Treatment | | | |
| Compound 1 | Fmr1 KO WT littermates | 10 | 22.6† |
| WT control | (C57BL/6 bckgnd) | | |
| Compound 1 | Fmr1 KO (C57BL/6 bckgnd) | 10 | 22.6† |
| Treatment | | | |

### Results

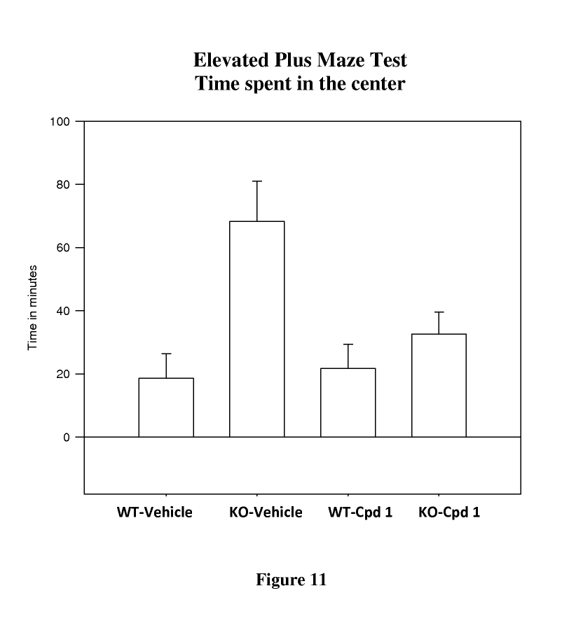

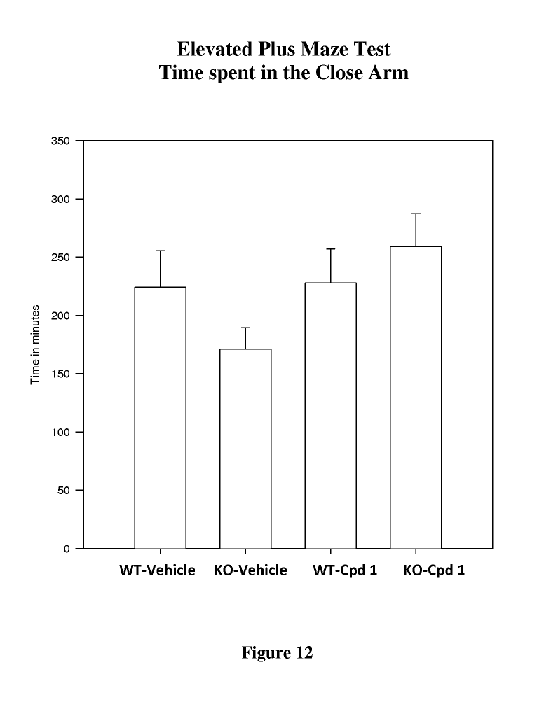

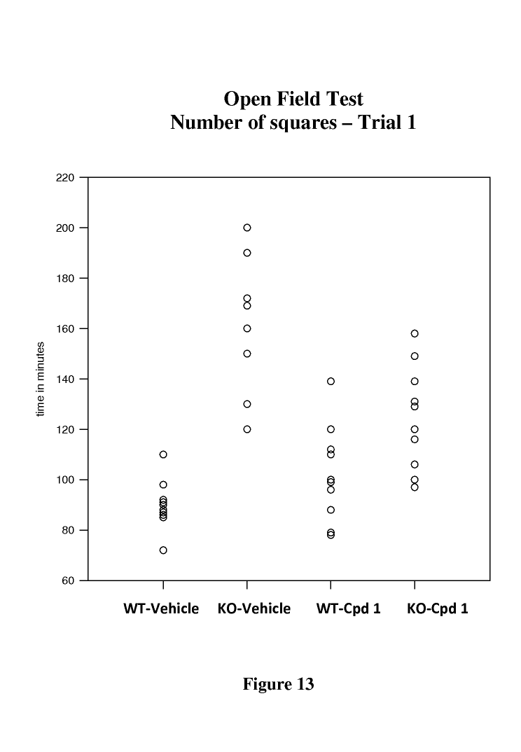

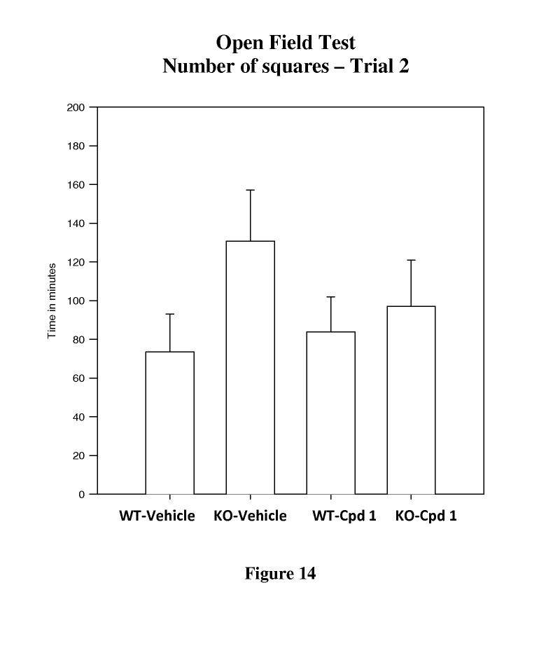

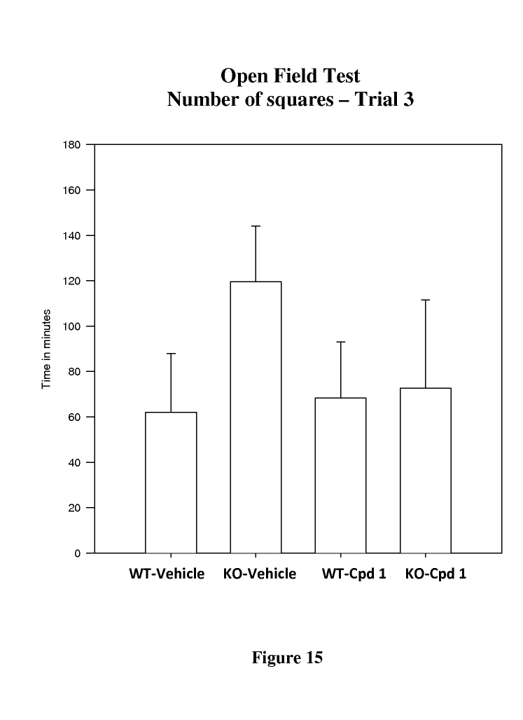

At baseline, Fmr1 KO mice manifested numerous phenotypic changes compared with wild-type littermate control mice, including hyperactivity in the open-field (p<0.01) and elevated plus maze test (p<0.01), Open Flield short and long term memory, and activities of daily living. Treatment with Compound 1 significantly ameliorated these aberrant features of the Fmr1 KO2 mouse phenotype. The results of the tests are presented in FIGS. 11-15 and in the Tables 13-16.

TABLE 13
Results of Elevated Plus Maze test: time spent in the close arm.

| Group Name | N | Missing | Mean time (min) | Std. dev. | SEM |
|---|---|---|---|---|---|
| WT-vehicle | 10 | 0 | 224.400 | 31.163 | 9.855 |
| KO-vehicle | 10 | 0 | 171.100 | 18.150 | 5.740 |
| WT-Compound 1 | 10 | 0 | 227.800 | 29.298 | 9.265 |
| KO-Compound 1 | 10 | 0 | 259.100 | 28.085 | 8.881 |

TABLE 14
Results of Open Field Test - Trial 2.

| Group Name | N | Missing | Mean time (min) | Std. dev. | SEM |
|---|---|---|---|---|---|
| WT-vehicle | 10 | 0 | 73.600 | 19.523 | 6.174 |
| KO-vehicle | 10 | 0 | 130.800 | 26.246 | 8.300 |
| WT-Compound 1 | 10 | 0 | 83.900 | 18.021 | 5.699 |
| KO-Compound 1 | 10 | 0 | 97.100 | 23.923 | 7.565 |

TABLE 15
Results of Open Field Test - Trial 3.

| Group Name | N | Missing | Mean time (min) | Std. dev. | SEM |
|---|---|---|---|---|---|
| WT-vehicle | 10 | 0 | 62.100 | 25.779 | 8.152 |
| KO-vehicle | 10 | 0 | 119.600 | 24.441 | 7.729 |
| WT-Compound 1 | 10 | 0 | 68.300 | 24.689 | 7.807 |
| KO-Compound 1 | 10 | 0 | 72.600 | 38.911 | 12.305 |

For the data presented in Tables 13-15 above, the differences in the mean values among the treatment groups are greater than would be expected by chance; there is a statistically significant difference (P<0001).

### Marble Burying Test

In the marble burying test, the Fmr1 KO2 mice buried significantly fewer marbles than wild type mice ((P<0001); this was significantly rescued by Compound 1, similar to vehicle-treated WT mice at all test sessions.

TABLE 16
Number of marbles buried out of 10 (median ± IQR).

| Group Name | N |
|---|---|
| WT-vehicle | 10 |
| KO-vehicle | 10 |
| WT-Compound 1 | 10 |
| KO-Compound 1 | 10 |

p-values: p (for vehicle treated mice) < 0001; p (for Compound 1 treated mice) < 0.005

### Contextual Fear Conditioning

Freezing as a species-specific response to fear was measured. Under acute stress conditions, the Fmr1 KO2 mice treated with Compound 1 failed to fully rescue the learning deficit, and exhibited a higher percentage of freezing as compared to the Compound 1 treated and vehicle treated WT mice.

### Conclusions

Overall, the results provide direct evidence that Compound 1 has a significant positive effect on hyperactivity, long and short term memory and species-typical behavior in the Fmr1 KO2 mice.

### Example 16

The purpose of this study was to investigate the direct effects of tetracycline compounds, such as minocycline and Compound 1, on dendritic spine development in the cell derived from the mouse model of Fragile X Syndrome (FXS) and their wild-type litter mate control mouse embryos.

### Materials and Methods

The compartmentalized cell culture was used in the experiments. Neuronal primary cultures of the hippocampus at embryonic day 16 (E16) were prepared from Fmr1 KO and WT litter mate control mouse embryos, and three independent cultures were used for the analysis. The in vitro system with GRP was used to monitor dendritic spine morphogenesis during a time-course of culture, and immunostaining with synaptophysin was used to distinguish presynaptic boutons. The dendritic spines were usually formed between 7 and 14 days in vitro (DIV). By 14 DIV most dendiritic protrusions were spines; however, their maturation continued until 21 DIV. The effects of tetracycline compounds were evaluated at 18 DIV. Confocal Imagining analysis was performed at the University of Chile imaging center. Quantitative analysis of dentritic spine length, size distribution for dendritic spine heads and morphology of the hippocampal neurons from WT control and Fmr1 KO mice was performed after 17 hour treatment with 20 μM tetracycline compounds or with PBS control.

### Results

Treatment with Compound 1 caused a significant reduction in spine number between FX hippocampal primary cultures (0.28±0.05) and controls (0.25±0.08).

### EQUIVALENTS

Those skilled in the art will recognize, or be able to ascertain using no more than routine experimentation, many equivalents to the specific embodiments and methods described herein. Such equivalents are intended to be encompassed by the scope of the present invention.

All patents, patent applications, and literature references cited herein are hereby expressly incorporated by reference.

What is claimed is:

1. A method for treating or preventing a neurodegenerative disorder in a subject, the method comprising administering to said subject an effective amount of a tetracycline compound, or a pharmaceutically acceptable salt thereof, such that said neurodegenerative disorder is treated or prevented.

2. The method of claim 1, wherein the tetracycline compound is a compound of formula (I) 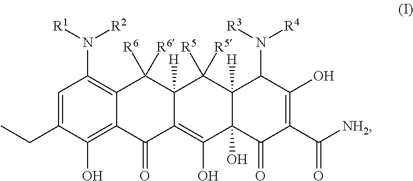
wherein:
R¹, R², R³ and R⁴ are each independently H or unsubstituted C₁-C₆ alkyl; and
R⁵, R^{5′}, R⁶ and R^{6′} are each independently H, hydroxyl, or unsubstituted C₁-C₆ alkyl.

3. The method of claim 2, wherein the tetracycline compound is a compound of formula (Ia) or (Ib):

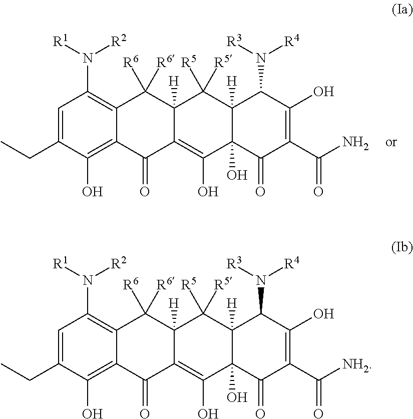

4. The method of claim 3, wherein the tetracycline compound is Compound 1:

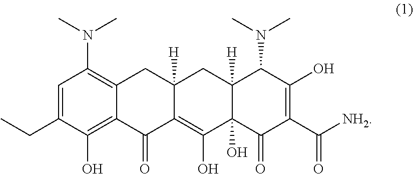 [SMILES: CCc1cc(N(C)C)c2c(c1O)C(=O)C1=C(O)[C@]3(O)C(=O)C(C(N)=O)=C(O)[C@@H](N(C)C)[C@@H]3C[C@@H]1C2]

5. The method of claim 1, wherein the neurodegenerative disorder is associated with inflammation of the brain.

6. The method of claim 5, wherein the neurodegenerative disorder is multiple sclerosis.

7. The method of claim 5, wherein the neurodegenerative disorder is autoimmune encephalomyelitis.

8. The method of claim 1, wherein the neurodegenerative disorder is a demyelination associated disorder.

9. The method of claim 8, wherein the demyelination associated disorder is multiple sclerosis.

10. The method of claim 8, wherein a dosage of the tetracycline compound effective for inhibiting demyelination is lower than a dosage of minocycline effective for achieving the same extent of demyelination inhibition.

11. The method of claim 8, wherein axon loss is inhibited.

12. The method of claim 1, wherein the neurodegenerative disorder is stroke.

13. The method of claim 1, wherein the neurodegenerative disorder is Fragile X Syndrome.

14. A method for treating or preventing inflammation in a subject, the method comprising administering to said subject an effective amount of a tetracycline compound, or a pharmaceutically acceptable salt thereof, such said inflammation is treated or prevented.

15. The method of claim 14 wherein the tetracycline compound is a compound of formula (I) 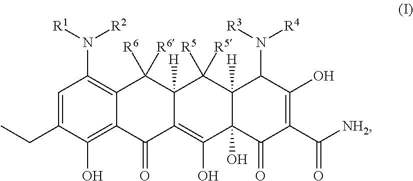
wherein:
R¹, R², R³ and R⁴ are each independently H or unsubstituted C₁-C₆ alkyl; and
R⁵, R^{5′}, R⁶ and R^{6′} are each independently H, hydroxyl, or unsubstituted C₁-C₆ alkyl.

16. The method of claim 15, wherein the tetracycline compound is a compound of formula (Ia) or (Ib):

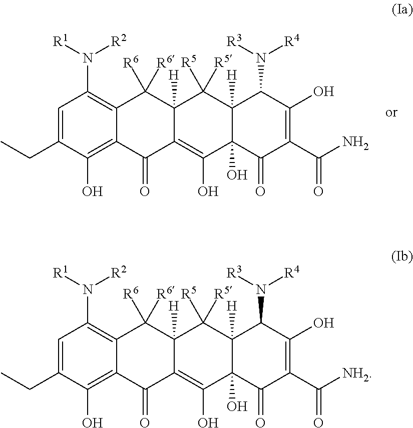

17. The method of claim 16, wherein the tetracycline compound is Compound 1:

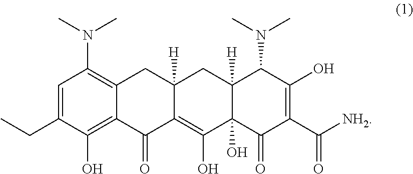 [SMILES: CCc1cc(N(C)C)c2c(c1O)C(=O)C1=C(O)[C@]3(O)C(=O)C(C(N)=O)=C(O)[C@@H](N(C)C)[C@@H]3C[C@@H]1C2]

18. The method of claim 14, wherein MMP-9 activity is inhibited.

19. The method of claim 14, wherein TNFα is inhibited.

20. The method of claim 14, wherein nitric oxide production by macrophages is inhibited.

21. A method for treating or preventing stroke in a subject, the method comprising administering to said subject an effective amount of Compound 1, or a pharmaceutically acceptable salt thereof, such that stroke is treated or prevented.

22. A method for treating or preventing multiple sclerosis in a subject, the method comprising administering to said subject an effective amount of Compound 1, or a pharmaceutically acceptable salt thereof, such that multiple sclerosis is treated or prevented.

23. A method for treating Fragile X Syndrome in a subject, the method comprising administering to said subject an effective amount of Compound 1, or a pharmaceutically acceptable salt thereof, such that Fragile X Syndrome is treated.

24. The method of any one of claim 21, 22 or 23, wherein the subject is a human.

25. The method of claim 1, wherein the neurodegenerative disorder is treated with less tissue staining than caused by the same dose of minocycline.

26. The method of claim 25, wherein the neurodegenerative disorder is treated without substantial tissue staining.

27. The method of claim 1, wherein the neurodegenerative disorder is treated with lesser antibacterial effect than caused by the same dose of minocycline.

28. The method of claim 27, wherein the neurodegenerative disorder is treated without substantial antibacterial activity.

29. The method of claim 26 or 28, wherein the neurodegenerative disorder is multiple sclerosis.

30. The method of claim 1, wherein the neurodegenerative disorder is encephalomyelitis.
